(19) United States
(12) Patent Application Publication
(10) Pub. No.: US 2025/0339051 A1
(43) Pub. Date: Nov. 6, 2025

(54) DYNAMIC CHEMICAL-EXCHANGE-SATURATION-TRANSFER (CEST) AGENT ENHANCED MRI USING DIRECT WATER SATURATION

(71) Applicants: The Johns Hopkins University, Baltimore, MD (US); KENNEDY KRIEGER INSTITUTE, INC., Baltimore, MD (US)

(72) Inventors: Peter VAN ZIJL, Hanover, MD (US); Linda KNUTSSON, Baltimore, MD (US); Nirbhay N. YADAV, Baltimore, MD (US)

(73) Assignees: The Johns Hopkins University, Baltimore, MD (US); KENNEDY KRIEGER INSTITUTE, INC., Baltimore, MD (US)

(21) Appl. No.: 19/196,099

(22) Filed: May 1, 2025

Related U.S. Application Data
(60) Provisional application No. 63/641,180, filed on May 1, 2024.

(51) Int. Cl. A61B 5/055; A61B 5/00; A61B 5/145

(52) U.S. Cl. CPC A61B 5/055; A61B 5/0075; A61B 5/14532; A61B 5/14546

(57) ABSTRACT

A method for at least one of magnetic resonance (MR) imaging (MRI) or spectroscopy (MRS) on an MR scanner for detecting the presence of a changed amount of a substance containing exchangeable protons in one or more tissue areas in a human or non-human subject includes subjecting the subject to an MR procedure capable of generating MR signals encoding at least one tissue area in the subject in which the amount of the substance is changing; acquiring at least one water saturation spectrum (Z-spectra) with a substantial direct water saturation (DS) component in the subject before and after a change in the amount of the substance; detecting at least one of a tissue-based or temporal variation in a width, a shape, a frequency, or an integral of the DS component as a consequence of the change in the amount of the substance; determining at least one tissue-related parameter from the tissue-based or temporal variation; and ascertaining whether the at least one tissue-related parameter is abnormal.

This application claims priority to U.S. Provisional Application No. 63/641,180, filed May 1, 2024, which is incorporated herein by reference in its entirety.

### FEDERAL FUNDING

This invention was made with government support under grants EB019934 and EB034978 awarded by the National Institutes of Health. The government has certain rights in the invention.

### BACKGROUND

### 1. Technical Field

The currently claimed embodiments of the present invention relate to magnetic resonance imaging (MRI) and/or magnetic resonance spectroscopy (MRS) systems and methods, and more particularly to MRI and/or MRS systems and methods for dynamic chemical-exchange-saturation-transfer (CEST) agent enhanced MRI and/or MRS using direct water saturation.

### 2. Discussion of Related Art

Inventors of the current application previously patented the use of non-labeled sugars and their detection by MRI for assessing tissue perfusion and metabolism (patents U.S. Pat. Nos. 9,180,211 and 10,967,076 which are incorporated herein in their entirety). The ability to use sugars or other biodegradable CEST agents instead of metallic agents for MRI, would provide improvement of care for the 12,000,000 patients who receive Gadolinium-based contrast agents (GBCAs) in the USA alone. Over the past decade, several research groups have shown the possibility of using sugars as contrast agents in animal models of, for instance, cancer and neurodegenerative disease (for a recent review, see: Knutsson L, Xu X, van Zijl P C M, Chan K W Y. Imaging of sugar-based contrast agents using their hydroxyl proton exchange properties. NMR Biomed. 2023 June; 36(6):e4784 which are incorporated herein by reference). However, while these methods work well at high magnetic field (≥7 T), even in humans, investigators have run into a problem regarding the small magnitude of the MRI signal change (on the order of 1% of the water signal) at commonly used clinical magnetic field strengths (B0), such as 3 Tesla (3 T) and 1.5 T, leading to inaccuracies in detection, especially when motion is present. Currently used technologies include measurement of MRI signal intensities or integrals employing chemical exchange saturation transfer (CEST) or chemical exchange spin lock (CESL) imaging. These MRI signals are affected by the presence of sugars through exchange of protons between the hydroxyl protons in sugars and the water protons used for MRI detection. CESL is based on changes in T1 relaxation in the rotating frame (T1rho). T2 is also sensitive to exchange. All of these methods were included in the previous patents, CESL and T1p and T2 imaging fall under claim 15 in U.S. Pat. No. 9,180,211 and claim 1, last paragraph, in U.S. Pat. No. 10,967,076 which are incorporated herein by reference. There thus remains a need for improvements in such MRI and/or MRS systems and methods.

### SUMMARY

A method for at least one of magnetic resonance (MR) imaging (MRI) or spectroscopy (MRS) on an MR scanner for detecting the presence of a changed amount of a substance containing exchangeable protons in one or more tissue areas in a human or non-human subject according to an embodiment of the current invention includes subjecting the subject to an MR procedure capable of generating MR signals encoding at least one tissue area in the subject in which the amount of the substance is changing; acquiring at least one water saturation spectrum (Z-spectra) with a substantial direct water saturation (DS) component in the subject before and after a change in the amount of the substance; detecting at least one of a tissue-based or temporal variation in a width, a shape, a frequency, or an integral of the DS component as a consequence of the change in the amount of the substance; determining at least one tissue-related parameter from the tissue-based or temporal variation; and ascertaining whether the at least one tissue-related parameter is abnormal.

A computer-readable medium according to an embodiment of the current invention includes non-transient computer executable code, which when executed on a computer, causes said computer to perform a method according to an embodiment of the current invention.

An MRI or MRS system according to an embodiment of the current invention includes a processor comprising that is configured to perform a method according to an embodiment of the current invention.

### BRIEF DESCRIPTION OF THE DRAWINGS

Further objectives and advantages will become apparent from a consideration of the description, drawings, and examples. The patent or application file contains at least one drawing executed in color. Copies of this patent or patent application publication with color drawings will be provided by the Office upon request and payment of the necessary fee.

FIG. 1 is a schematic illustration of a system for magnetic resonance imaging according to some embodiments.

FIG. 2A shows clinical MRIs (Gd-enhanced T1w, FLAIR, corrected cerebral blood volume (CBV) and K^{trans} images) together with line width (LW) and area under the curve (AUC) direct saturation dynamic glucose enhanced (DS-DGE) images for a patient with a diffuse astrocytoma. Corrected CBV (increased blood volume) and K^{trans} indicate presence of malignant tumor lateral to the ventricle.

FIG. 2B (top row): DS-DGE AUC also shows enhancement in the areas indicative of tumor at corrected CBV and K^{trans}, but in addition in a tumor region not enhanced with Gd. This possibly indicates early breakdown of the blood brain barrier (BBB) in other regions that Gd enhancement is not capable of detecting; (bottom row) After treatment the images show disappearance of these regions in K^{trans} and DS-DGE, but not corrected CBV.

FIG. 2C shows region of interest analysis for linewidth changes (ΔLW) as a function of time (orange is the infusion period) in the DS spectrum of tumor regions before treatment and the same regions after treatment.

FIG. 3A shows clinical MRIs (Gd-enhanced T1w, FLAIR, corrected CBV, K^{trans} and extracellular extravascular volume (Ve) images) and LW and DS-DGE AUC images for a patient with a lung cancer metastasis. Corrected CBV shows no increased blood volume, while K^{trans} and an increased Ve show a clear breakdown of the BBB, typical for metastasis. Additionally, the DS-DGE AUC image shows strong enhancement. Signal in the contralateral hemisphere is also enhanced due to presence of a ventricular lobe.

FIG. 3B shows region of interest analysis for linewidth changes as a function of time (orange is the infusion period) in the DS spectrum (ΔLW) for the tumor region and a white matter region.

FIG. 4 shows simulations of DGE difference Z-spectrum as a function of static field strength (11.7, 7.0, and 3.0 T) in different tissue compartments. Four different RF powers from 0.4 to 2.0 μT are simulated for a saturation time of 1 second. The simulation assumes that blood glucose concentration increase from 5 to 15 mM after infusion and the glucose in other tissue compartments is one-quarter of the blood concentration. The plots for the GM and WM are enlarged in the black box below. From: Xu X, Sehgal A A, Yadav N N, Laterra J, Blair L, Blakeley J, Seidemo A, Coughlin J M, Pomper M G, Knutsson L, van Zijl P C M. d-glucose weighted chemical exchange saturation transfer (glucoCEST)-based dynamic glucose enhanced (DGE) MRI at 3 T: early experience in healthy volunteers and brain tumor patients. Magn Reson Med. 2020 July; 84 (1): 247-262.

FIG. 5 shows simulations of compartmental and whole-brain D-glucose concentrations for gray matter (GM, left column), white matter (WM, center column) and glioblastoma (GBM, right column) calculated using a 3-compartment model with endothelial cells in the BBB (EN). Compartmental D-glucose concentrations for endothelium, C_{EN}(t), extravascular extracellular space (EES), Cₑ(t), tissue cells, C_{c}(t), as well as tissue glucose-6-phosphate, C_{G6P}(t), were calculated from the arterial plasma input concentration,

$${C}_{p}^{a} ( t ) ,$$

which was 6.15 mM. Before infusion, the D-glucose concentration is 1.08. 1.54, and 4.61 mM in the EES for GM, WM, and GBM, respectively. Notice that the initial baseline is not flat in the graphs. The reason is that no constant baseline start value was used, but rather the model was applied using the Michaelis Menten constants, thus it takes time for the system to reach equilibrium. A baseline of 45 min was used to accomplish this.

FIG. 6: Patient with IDH-wildtype glioblastoma, MGMT unmethylated. (Top left) Anatomical images (Gd-T₁w and FLAIR). (Middle left) corrected CBV and K2 trans (leakage rate) from DSC MRI. (Bottom left) K^{trans} and Vₑ from DCE MRI. (Top right) Left shows a parametric map of the MTR obtained by integrating the MTR_{asym} from 1.0 to 2.2 ppm, which is the asymmetry in the DS (DS_{asym}). Z-spectra obtained from ROIs placed in the DS_{asym} contrast-enhanced lesion area, corresponding to the enhancement in the Gd-T1w image, and NAWM (4 ppm to −4 ppm). (Bottom right) Graph of the corresponding DS_{asym} (MTR_{asym}) obtained from the ROIs placed in the lesion and the NAWM (4 ppm to 0 ppm). The data show FLAIR hyperintensity and a contrast-enhancing rim on the Gd-T1w image. K2, corrected CBV, Vₑ and K^{trans} all show an increase corresponding to the contrast-enhanced area on the Gd-T1w. The DS_{asym} image displays a hyperintense region in in the tumor region, corresponding approximately to the hyperintense FLAIR signal and the contrast enhancing area in the Gd-T1w image. The DS_{asym} measured in the ROIs was higher in the lesion than in the NAWM. The maximum value of DS_{asym} in the tumor lesion was close to 0.03, located at 1.9 ppm. Abbreviations: corr. CBV, corrected cerebral blood volume; DCE, dynamic contrast enhanced; DSC, dynamic susceptibility contrast; FLAIR, fluid-attenuated inversion recovery; Gd, gadolinium; K2, leakage; K^{trans}, volume transfer constant; MTR_{asym}, magnetization transfer ratio with asymmetric analysis; NAWM, normal-appearing white matter; ROI, regions-of-interest; T₁w, T₁ weighted; Vₑ, interstitial volume.

FIG. 7: Same patient as FIG. 6, with IDH-wildtype glioblastoma, MGMT unmethylated. Dynamic glucose enhanced (DGE) signal maps and curves. (left) Parametric map of the DGE MTR_{asym} obtained by first integrating the MTR_{asym} from 1.0 to 2.2 ppm through all dynamics and then subtracting each dynamic with the average integrated MTR_{asym} baseline. The result is then integrated over time to get the area under the curve (AUC) map. Also shown is the ΔMTR_{asym} time curve obtained from ROIs placed in the MTR_{asym} contrast-enhanced lesion area (excluding regions from the cavities as determined from hypointensity in the Gd-T1). The ΔMTR_{asym} time curve was calculated by subtracting each integrated MTR_{asym} dynamic from the average integrated MTR_{asym} baseline. Shown at the bottom are MTR_{asym} curves obtained from the ROIs placed in the same area as above. Blue shows the average integrated MTR_{asym} at baseline and orange shows the average integrated MTR_{asym} between 15 to 20 minutes postinfusion. The maximum of the green curve occurs at a frequency corresponding approximately to the frequency where the maximum of the combined signals of the multiple OH resonances of D-glucose is expected to appear. (Right) Parametric map of the DGE AUC obtained by first integrating the Z-spectra and then taking the difference of the average infusion Z-spectra and baseline infusion Z-spectra (AZ-spectra). Also shown is the ΔZ-spectra time curve obtained from ROIs placed in the MTR_{asym} contrast-enhanced lesion area (excluding the hypointensity from the cavities in the Gd-T1). Shown at the bottom is the subtraction between average baseline Z-spectra and the average Z-spectra between 15 to 20 minutes postinfusion. Abbreviations: MTR_{asym}, magnetization transfer ratio with asymmetric analysis

FIG. 8 shows normoglycemic

$$( {C}_{p}^{a} = 6.15 mM )$$

and hyperglycemic

$$( {C}_{p}^{a} = 19.8 mM )$$

simulated Z-spectra for tissue compartments (left) and total tissue (right). The lower row shows a zoomed-in view comparing the Z-spectral intensities around half-maximum for normo- and hyperglycemia. Only Z-spectra with a sufficiently large change are visualized for tissue compartments (lower left). Saturation parameters: B₁ₚₑₐₖ=0.5 μT, ten consecutive 50-ms sinc-gauss pulses for tₛₐₜ=0.5 s (TR=1.2 s).

FIG. 9 shows patient with recurrent IDH-wildtype glioblastoma showing thin Gd-enhancement around the resection cavity. (Left) ΔLW maps during the scan (averaged over a period of 76 s corresponding to two ΔLW images). (Right top) Anatomical images (Gd-T₁w, FLAIR), together with parametric maps from DCE MRI (K^{trans}, Vₑ), DSC MRI (corrected CBV, K2) and DS-DGE MRI (AUC grayscale and color-coded). (Right bottom) Graphs of linewidth change vs time obtained from ROIs placed in the DS-DGE peri-cavity infiltrative tumor region, located anterior to the cavity, and ventricle (purple diamonds and blue squares, respectively). The ROIs are overlayed on the DS-DGE MRI AUC map located in the graph as purple and blue areas, respectively. To visualize the trend in glucose uptake, ΔLW(t) curves were temporally smoothed with a 3-point moving average (purple and blue lines).

FIG. 10 shows (Left) Normoglycemic (ngl) and hyperglycemic (hgl) experimental Z-spectra from the glioblastoma patient in FIG. 9 and the corresponding Deep-Learning (DL) based Lorentzian fits. (Right) A zoomed-in view demonstrating the linewidth difference between normo- and hyperglycemic experimental Z-spectra. ROI locations are displayed in the Gd-T₁w image and DS-DGE AUC map.

FIG. 11 shows patient with grade 2 IDH-mutated Astrocytoma. Anatomical images (Gd-T₁w, FLAIR) together with corrected CBV, K2, K^{trans}, Vₑ, and DS-DGE MRI maps (AUC in both grayscale and color-coded). Color-coded AUC calculated from the infusion block only is also shown. A DS-DGE AUC map overlayed on fused Gd-T₁w/FLAIR is shown for reference. Graph of linewidth change vs time obtained from ROI placed in the DS-DGE contrast-enhanced area (purple overlayed on DS-DGE AUC map). To visualize the trend in glucose uptake, ΔLW(t) curves (purple dots) were temporally smoothed with a 3-point moving average (purple line).

FIG. 12 shows patient with a grade 2 IDH-mutated astrocytoma. Anatomical images (Gd-T1w, FLAIR) together with parametric maps from DCE MRI (K^{trans}, Vₑ), DSC MRI (uncorrected CBV, corrected CBV, K2) and DS-DGE MRI (color-coded AUC) from four slices.

FIG. 13 shows patient with brain metastasis from ALK-mutated non-small-cell lung cancer. Anatomical images (Gd-T1w, FLAIR) together with K^{trans}, Vₑ, uncorrected CBV, corrected CBV, and DS-DGE MRI maps (LW map from 3^{rd} dynamic, AUC maps in both grayscale and color-coded). A color-coded AUC calculated from the infusion block only is also shown. A graph of linewidth change vs. time obtained from ROIs placed in the DS-DGE contrast-enhanced area and contralateral frontal WM is shown to the right (purple dots and orange diamonds, respectively). ROIs are overlayed on the DS-DGE MRI AUC map in the graph as purple and orange areas, respectively. To visualize the trend in glucose uptake, ΔLW(t) curves were temporally smoothed with a 3-point moving average (purple and orange lines).

### DETAILED DESCRIPTION

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The below-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. All references cited anywhere in this specification, including the Background and Detailed Description sections, are incorporated by reference as if each had been individually incorporated.

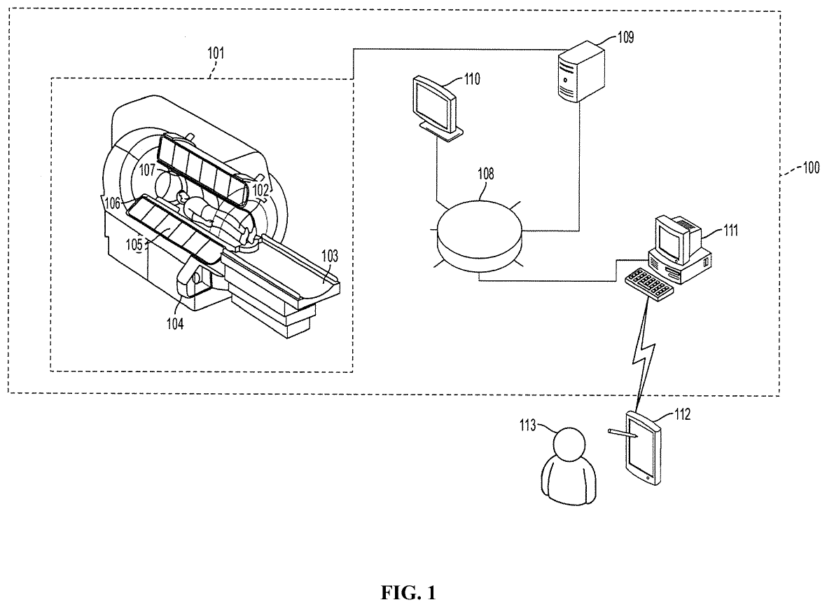

FIG. 1 shows a system 100 for magnetic resonance imaging according to some embodiments of the current invention. The system 100 includes an MRI system 101. The MRI system 101 can accommodate a subject 102 under observation on scanner bed 103, for example. The MRI system 101 can include, but is not limited to, a primary magnet system 105 providing a substantially uniform main magnetic field B₀ for a sample (subject or object) 102 under observation on scanner bed 103, a magnetic gradient coil system 106 providing a perturbation of the main magnetic field B0 to encode spatial information of the constituent molecules of subject 102 under observation, and a radiofrequency (RF) coil system 107 to transmit electromagnetic waves and to receive magnetic resonance signals from subject 102. The RF coil system 107 may include separate radiofrequency transmit and receive coils, each having a plurality of coils. For instance, receivers can have multiple MRI detectors, such as those provided in an ‘MRI phased-array.’ Some embodiments of the invention include 16, 32, 60, or 120 MRI detectors, though these numbers are provided as examples, and the embodiments of the invention are not limited to these examples. Each MRI detector has a spatial sensitivity map.

The system 100 also has a processor 109 configured to communicate with the MRI system 101. The processor 109 can be partially or totally incorporated within a structure 104 that houses the NMR system 101 and/or partially or totally incorporated in a computer, a server, or a workstation that is structurally separate from and in communication with the NMR system 101.

The system 100 can include a data storage unit 108 that can be, for example, a hard disk drive, a network area storage (NAS) device, a redundant array of independent disks (RAID), a flash drive, an optical disk, a magnetic tape, a magneto-optical disk, or that provided by local or remote computer ‘cloud’ networking, etc. However, the data storage unit 108 is not limited to these particular examples. It can include other existing or future developed data storage devices without departing from the scope of the current invention.

The processor 109 can be configured to communicate with the data storage unit 108. The processor 109 can also be in communication with a display system 110 and/or a console station 111. In some embodiments, results can be displayed by the display system 110 or the console station 111. In some embodiments, an operator 113 may use an input/output device 112 to interact, control, and/or receive results from system 100.

The MRI system 101 is configured to apply a plurality of spatially localized MRI sequences, wherein each sequence is adjusted to be sensitive to an MRI parameter whose measurement requires the acquisition of a plurality of spatially localized MR signals. The MRI system 101 is configured to adjust at least one of the applied plurality of spatially localized MRI sequences so as to substantially fully sample an image k-space of the sample, and adjust the remainder of the applied plurality of spatially localized MRI sequences to under-sample the image k-space of the sample. The MRI system 101 is configured to acquire a plurality of image k-space MRI signal data sets, each responsive to the application of each of the plurality of spatially localized MRI sequences. The processor 109 is configured to estimate a sensitivity map of each of the plurality of MRI detectors using a strategy to suppress unfolding artefacts, wherein the strategy is based on data acquired from the substantially fully-sampled spatially localized MRI sequence. The processor 109 is configured to apply the estimated sensitivity maps to at least one of the image k-space MRI signal data sets to reconstruct a spatial image of MRI signals that are sensitive to the MRI parameter within a Support Region of the spatial image in which the sample resides.

According to some embodiments of the invention, the MRI system 101 and the processor 109 are associated by one of an Ethernet connection, a Wi-Fi connection, or by integration of the processor 109 into the MRI system 101.

According to some embodiments, the processor 109 is configured to reconstruct an image whose intensity is directly proportional to a spatial distribution of the MRI parameter within the sample 102, and the display system 110 or the console station 111 is configured to display the reconstructed image.

In other embodiments, the system 100 can be configured as an MRS system.

Here we describe two new acquisition approaches together with appropriate analysis approaches that have better sensitivity to measuring the small effects at lower magnetic fields that exist in clinical scanners according to some embodiments of the current invention. It is not applicable only to sugars, but also to the broader field of all CEST agents.

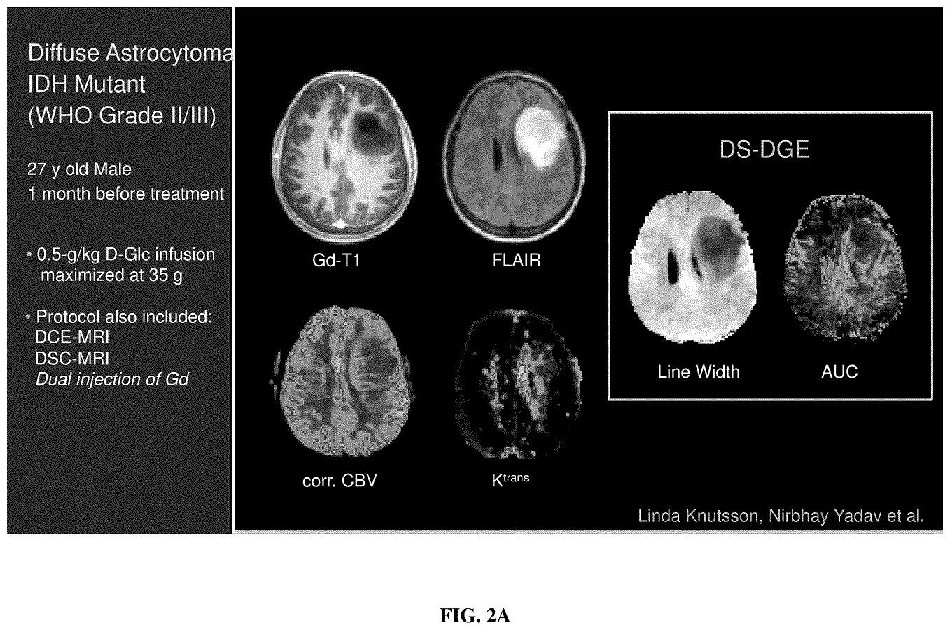

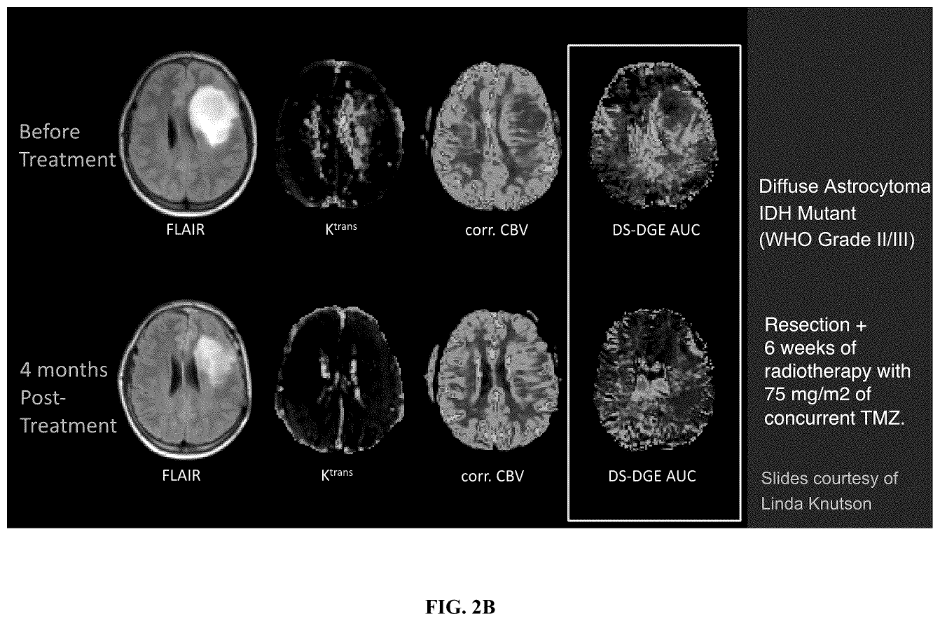

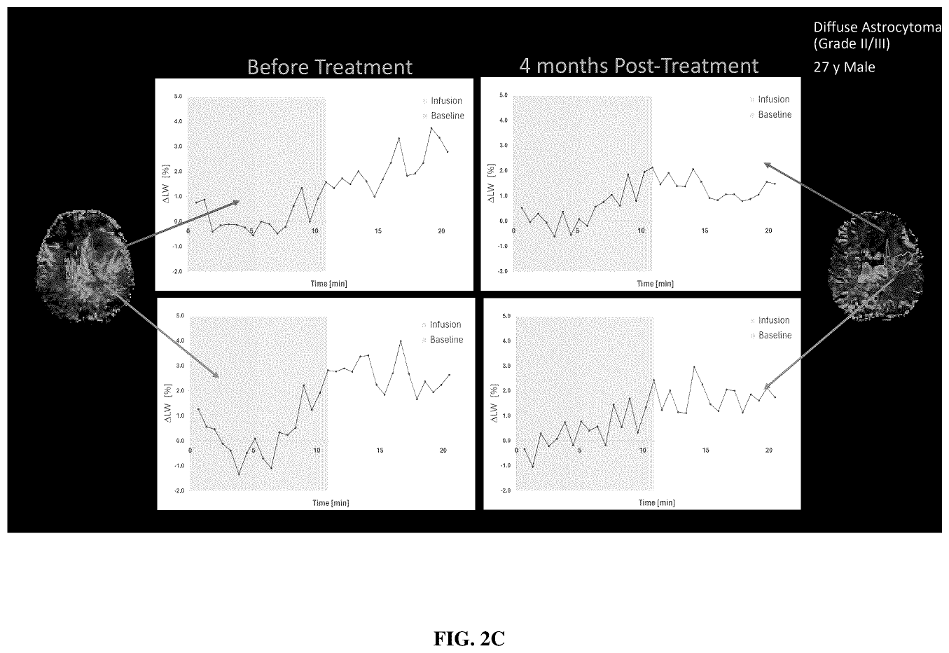

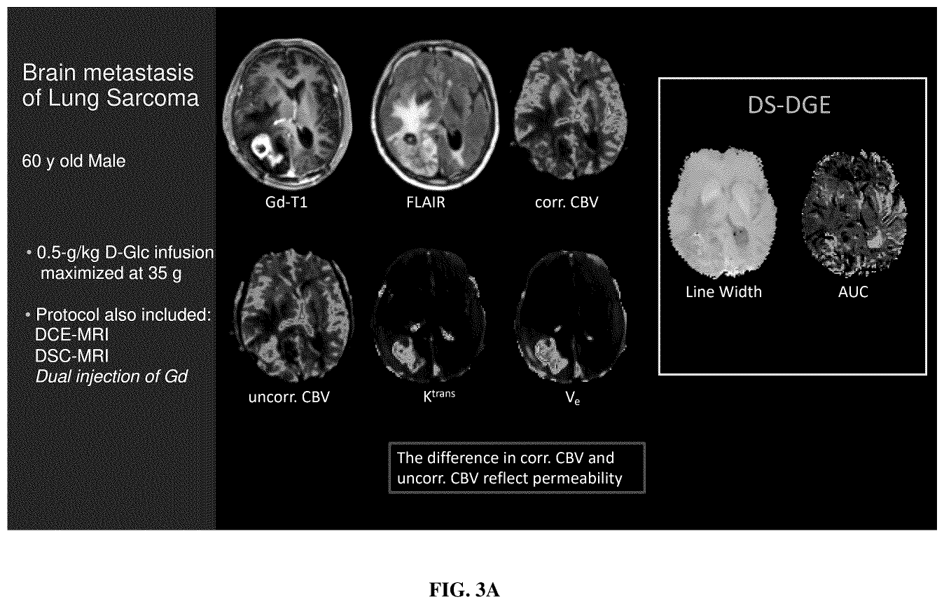

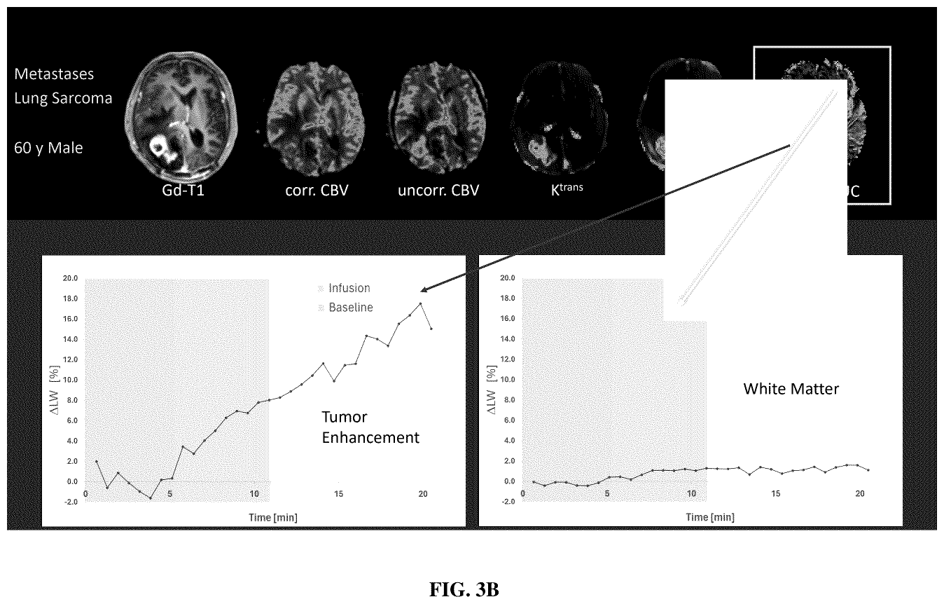

(i) The first is based on measuring the linewidth and changes therein of the direct water saturation signal (DS) in the Z-spectrum (Water saturation spectrum). This requires data acquisition as a function of irradiation frequency at sufficiently low radiofrequency field strengths (B1) and short saturation time (tsat) to avoid interference of background signals due to either semi-solid magnetization transfer effects or exchangeable proton signals from tissue molecules. Such acquisitions were covered in claims 1-7 of a previous technology of us for a different purpose (Frequency Referencing Method for Chemical Exchange Saturation Transfer (CEST) MRI, patent number U.S. Pat. No. 8,536,866, incorporated herein by reference). However, the purpose of that patent was to determine the water frequency, while here the goal is to determine the linewidth of the DS signal (which is a function of the relaxation time T2) and changes therein due to the change in glucose concentration in the volume element (voxel) in the image. This can be fitted for instance using a Lorentzian lineshape or similar functions or just measured after signal interpolation or machine learning or deep learning fitting of the shape. All methods of claim 7 of patent U.S. Pat. No. 8,536,866 can also be used. We have data showing that this approach works for brain tumor patients at 3 T. Below are some data in a diffuse astrocytoma (FIGS. 2A-2C) and a lung cancer metastasis (FIGS. 3A-3B).

(ii) Acquisitions using high-B1 (>=1.5 μT), and tsat >=0.3 s Z-spectral acquisitions to assess T2 effects on the DS signal together with the asymmetric broadening due to the intermediate to fast exchange condition for hydroxyl protons in sugars as well as amine protons in other sugars, e.g., such as glucosamine. This is followed by assessment of the asymmetry in the DS difference signal due to the changes in glucose signal concentration.

Below are some simulations from a paper published by us (Xu X, Sehgal A A, Yadav N N, Laterra J, Blair L, Blakeley J, Seidemo A, Coughlin J M, Pomper M G, Knutsson L, van Zijl P C M. d-glucose weighted chemical exchange saturation transfer (glucoCEST)-based dynamic glucose enhanced (DGE) MRI at 3 T: early experience in healthy volunteers and brain tumor patients. Magn Reson Med. 2020 July; 84 (1): 247-262, the entire content of which is hereby incorporated by reference) showing the D-glucose difference signal, but nobody has ever proposed to use the asymmetry (difference in intensity or integral between the left and the right of the water signal) to assess the glucose presence effect. We will also assess the total difference signal over the whole frequency spectrum, also not done previously.

### FURTHER EMBODIMENTS

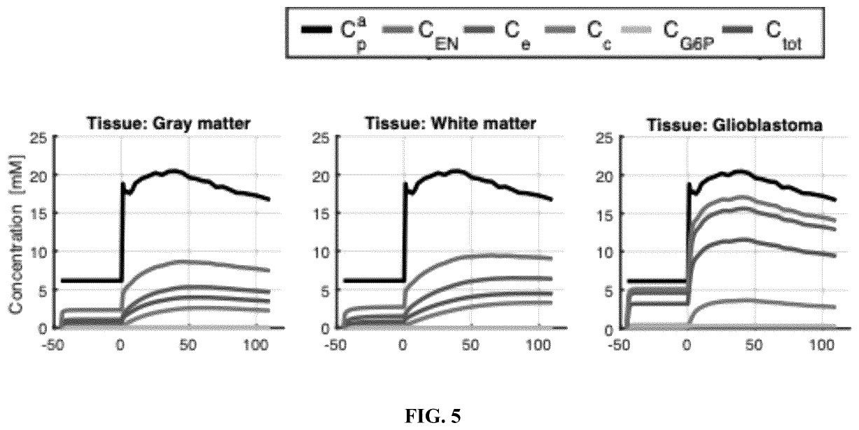

1) We derived a theory to describe sugar uptake, i.e. delivery, transport, and metabolism into tissue. When performing simulations for different tissue types using D-Glucose as a substrate, malignant tumors in which the blood brain barrier (BBB) has been broken show an increase in concentration of glucose in the extravascular extracellular space (EES) with about 3-4 mM (FIG. 5). Malignant tumor EES generally has an increased EES volume (Vₑ). In addition, EES in malignant tumors has a reduced pH, leading to reduced proton exchange. These combined effects lead to a significant change in the MR parameters described compared to normal tissue. This is especially the case for the asymmetry in the DS water line shape, which should become detectable as a contrast between tumor and normal gray and white matter.

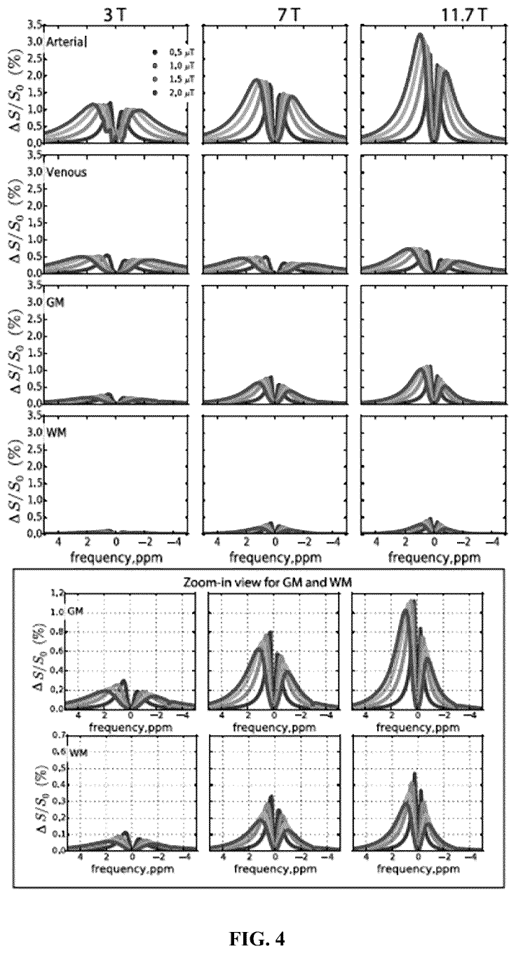

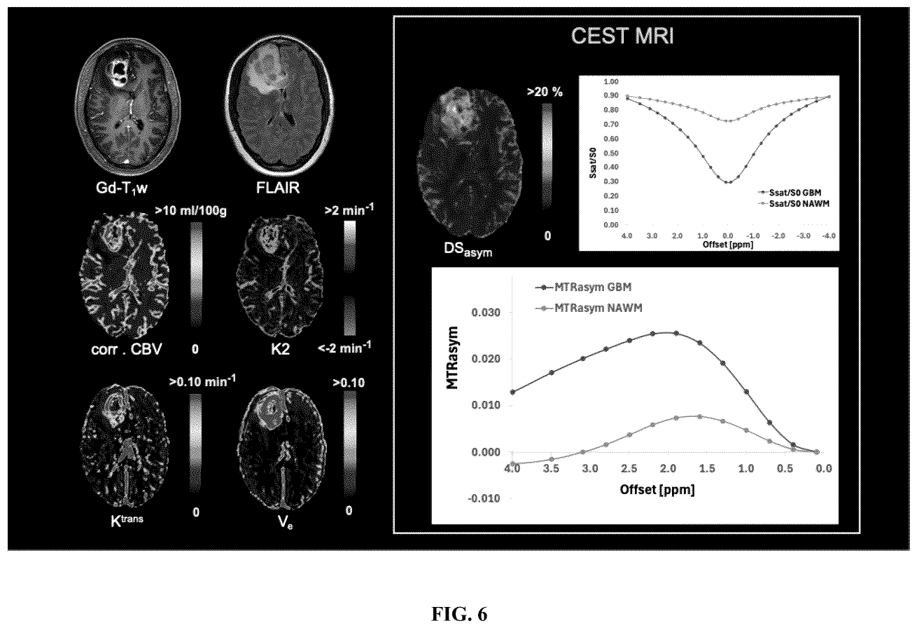

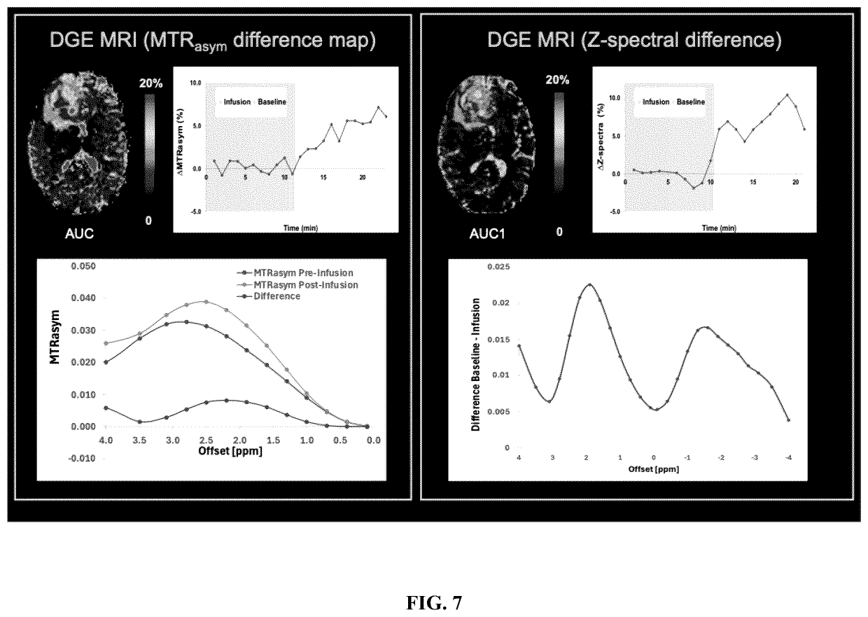

We have acquired new data with an increased B1 (2.0 μT and 0.5 s saturation time) and studied both glucose infusion and the baseline in recurrent tumors. FIG. 6 shows a case with a glioblastoma, where we analyzed the asymmetry relative to the DS lineshape at baseline (FIG. 6) and after infusion of 35 g of D-glucose (FIG. 7). Notice the difference between the two types of maps (larger enhancement region after infusion in FIG. 7), indicating that the glucose infusion points out regions of interest with BBB breakdown (something that may also be concluded from the delayed increase in signal difference compared to the time of infusion. As there is no fluid suppression used, the ventricles and cavities and blood vessels are also highlighted in these Figures. In FIG. 7 left versus right we see the difference map before and after infusion with taking the asymmetry and without taking the MTRasym, resp. The spectrum on the bottom right clearly resembles the simulations in FIG. 4.

### EXAMPLES

The following describes some further aspects of the current invention by way of some examples. The general concepts of the current invention are not limited to only these examples. In particular, the following describes: Purpose: Dynamic glucose enhanced (DGE) MRI studies employ chemical exchange saturation transfer (CEST) or spin lock (CESL) to study glucose uptake. Currently, these methods are hampered by low effect size and sensitivity to motion. To overcome this, we can utilize exchange-based linewidth (LW) broadening of the direct water saturation (DS) curve of the water saturation spectrum (Z-spectrum) during and after glucose infusion (DS-DGE MRI). Methods: To estimate the glucose-infusion-induced LW changes (ΔLW), Bloch-McConnell simulations were performed for normoglycemia and hyperglycemia in blood, gray matter (GM), white matter (WM), CSF, and malignant tumor tissue. Whole-brain DS-DGE imaging was implemented at 3 tesla using dynamic Z-spectral acquisitions (1.2 s per offset frequency, 38 s per spectrum) and assessed on four brain tumor patients using infusion of 35 g of D-glucose. To assess ΔLW, a deep learning-based Lorentzian fitting approach was employed on voxel-based DS spectra acquired before, during, and post-infusion. Area-under-the-curve (AUC) images, obtained from the dynamic ΔLW time curves, were compared qualitatively to perfusion-weighted imaging (PWI) parametric maps. Results: In simulations, ΔLW was 1.3%, 0.30%, 0.29/0.34%, 7.5%, and 13% in arterial blood, venous blood, GM/WM, malignant tumor tissue, and CSF, respectively. In vivo, ΔLW was approximately 1% in GM/WM, 5-20% for different tumor types, and 40% in CSF. The resulting DS-DGE AUC maps clearly outlined lesion areas. Conclusions: DS-DGE MRI can be useful for assessing D-glucose uptake. Example results in brain tumor patients show high-quality AUC maps of glucose-induced line broadening and DGE-based lesion enhancement similar and/or complementary to PWI.

### Introduction

Gadolinium (Gd) based contrast agents (GBCA) play a major role in MRI for both research and clinical routine. However, their use in certain patient groups is limited due to side effects such as nephrogenic systemic fibrosis^{1,2}. In vivo deposition³⁻⁵ has also led to the FDA issuing a Box Package Warning on GBCAs, which remain under continued review⁶. In addition, many malignant tumors show little to no Gd-enhancement^{7,8}. Consequently, current clinical practice is judicious use of GBCA, particularly in young and vulnerable populations⁹. Thus, there is a need to develop new contrast agents. The availability of chemical exchange saturation transfer (CEST)¹⁰⁻¹⁷ and chemical exchange sensitive spin lock (CESL)¹⁸⁻²⁹ approaches has opened the MRI field to non-metallic contrast agents. When D-glucose (D-Glc) is used as an agent, these approaches have been dubbed glucoCEST and glucoCESL, respectively.

Dynamic glucose enhanced (DGE) MRI applies CEST or CESL of sugars dynamically, providing information on contrast agent uptake in tissues in a manner similar to dynamic contrast-enhanced (DCE) and dynamic susceptibility contrast (DSC) MRI^{13,14,23-25,27,28,30-35}. Unfortunately, DGE MRI signal changes at clinical field strengths (3 T) have been on the order of one percent^{17,32}, resulting in sensitivity to motion artifacts, especially when based on a single signal intensity from one saturation offset frequency per dynamic^{36,37}. Here, we describe reducing these problems by utilizing the transverse relaxation effect originating from the chemical shift difference between the hydroxyl and water proton pools. The exchange between these pools leads to spins experiencing different precession frequencies^{38,39}, resulting in a collective phase dispersion and a linewidth (LW) broadening of the direct saturation (DS) line shape in water saturation spectra (Z-spectra). We exploit this exchange-based relaxation enhancement to assess changes in D-Glc concentration by acquiring DS spectra using RF saturation of short duration and low B₁. This approach is commonly used in the Water Saturation Shift Referencing (WASSR) method⁴⁰ for measuring B₀ shifts, since it minimizes contributions from semi-solid magnetization transfer contrast (MTC), CEST, and relayed nuclear Overhauser effects (NOEs)⁴¹. This allows fitting of the full DS spectrum to a Lorentzian⁴², a method recently optimized using deep learning (DL)⁴³.

This study, therefore, aimed to develop a WASSR-analogous DGE method to utilize glucose-induced increases in the DS linewidth, dubbed DS-DGE MRI. Its feasibility in different types of tissues at 3 T was investigated through simulations and for D-Glc infusions in brain tumor patients. The in vivo DS-DGE effect size was compared qualitatively with DCE- and DSC-MRI parametric maps to investigate whether similar and/or complementary information was obtained.

### Methods

### Simulations

Bloch-McConnell simulations of Z-spectra at 3 T before and after D-Glc infusion were performed using Pulseq-CEST⁴⁴. Five tissues were simulated: blood, gray matter (GM), white matter (WM), malignant tumor with blood-brain barrier (BBB) disruption (TUMOR), and CSF. Importantly, hydroxyl exchange properties and water transverse relaxation times may differ between tissue compartments, leading to different signal contributions. Therefore, following a recently established model⁴⁵, three tissue compartments were simulated within WM, GM and TUMOR, namely blood (b), extravascular extracellular space (EES), and cell (c). Within blood, we assumed arteriolar (a) and venular (v) compartments. A previous DGE MRI study reported a venous plasma blood glucose average increase of 9.8 mM (N=11) for a D-Glc dose of 25 g³⁰. Since our experiments used a 40% higher dose (35 g), we assumed a 13.7 mM increase in venous blood D-Glc concentration from normoglycemic baseline to hyperglycemia. The resulting compartmental D-Glc concentrations after transport⁴⁵ (Table 1) were used to calculate the Z-spectral intensities,

$$\frac{S ( Δω )}{{S}_{0}} ,$$

using the hydroxyl proton pours at 0.66, 1.28, 2.08, and 2.88 ppm⁴⁶. All compartments, except for TUMOR EES, were assumed to have a pH of 7.2, with hydroxyl proton exchange rates of 2900, 6500, 5200 and 14300 Hz, respectively⁴⁶, at 37° C. For TUMOR EES, a pH of 6.8 was assumed, leading to exchange rates of 1500, 3100, 2500 and 6000 Hz, respectively⁴⁶. Compartmental Z-spectra were simulated using 41 frequency offsets: ±[10, 5.0, 4.0, 3.0, 2.5, 2.0, 1.75, 1.5, 1.25, 1.0, 0.80, 0.65, 0.55, 0.48, 0.40, 0.33, 0.26 0.18, 0.11, 0.036, 0.0] ppm. Saturation was applied using ten consecutive 50-ms sinc-gaussian pulses, resulting in a total saturation time (tₛₐₜ) of 0.5 s. The simulations were performed using a B₁ₚₑₐₖ of 0.5 μT.

Z-spectra in GM, WM, and TUMOR were calculated by adding the normalized signal of blood, EES, and cell⁴⁵:

$$\frac{{S}_{t i s} ( Δ ω )}{{S}_{0}} = [ {f}_{b , tis} {ρ}_{{}_{b}}^{{}_{V / V}} \{ {f}_{a} \frac{{S}_{a} ( Δ ω )}{{S}_{0 a}} + {f}_{v} \frac{{S}_{v} ( Δ ω )}{{S}_{0 v}} \} + {f}_{e , tis} {ρ}_{e}^{V / V} \frac{{S}_{e , tis} ( Δ ω )}{{S}_{0 e}} + {f}_{c , tis} {ρ}_{c , tis}^{V / V} \frac{{S}_{c , tis} ( Δ ω )}{{S}_{0 c}} ] / {ρ}_{t i s}^{V / V} , \qquad ( 1 )$$

with f_{i,tis} being the volume fraction for compartment i in mL compartment/mL tissue (Table 1). Capillary blood was assumed to have fast deoxygenation, resulting in the blood compartment (b) consisting of only arteriolar (a) and venular (v) subcompartments⁴⁷ with volume fractions fₐ=0.3 and fᵥ=0.7 in mL/mL blood, respectively. Since MR contrast is determined by water-based compartmental concentrations and tissue volume fractions, corrections are included for ρ^{V/V}, the water content per mL compartment or tissue (Table 1). After adding the Z-spectra from each compartment, 2% Rician noise was applied. The resulting tissue Z-spectra were re-sampled at frequencies used in the experiments (28 frequencies from −5 to 5 ppm), followed by Lorentzian fitting using deep-learning⁴³. Thereafter, the linewidth difference (ΔLW) between normoglycemia (baseline) and hyperglycemia was calculated for each tissue using Eq. 2 below.

### Experiments

### Patients

Four brain tumor patients were studied (one with an IDH-wildtype glioblastoma, two with a grade 2 IDH-mutated astrocytoma, and one with ALK-mutated non-small-cell lung cancer metastases). The project was approved by the local Institutional Review Board, and each participant provided written informed consent. Participants were asked to fast six hours before the study, but clear liquids were permitted. Before the start of the MRI examination, blood was drawn to verify normal baseline glucose levels (3.9-7.0 mM). Table 2 lists the study's exclusion criteria.

### MRI Acquisition Protocol

Patients were examined on a 3 T Philips Elition RX system (Philips Healthcare, Best, the Netherlands). Pre- and post-contrast enhanced T₁-MPRAGE, FLAIR, DS-DGE, DCE and DSC images were acquired T₁-MPRAGE: TR/TE/FA=7.5 ms/3.5 ms/8°, FOV=212×212 mm², resolution=1.1×1.1×2.2 mm³, inversion time=755 ms, acquisition time=1 min 46 s, and FLAIR: TR/TE=11,000/120 ms, FOV=212×212 mm² resolution=1.1×1.1×2.2 mm³, inversion time=2800 ms, acquisition time=3 min 51 s.

DS-DGE images were acquired using ten consecutive 50-ms sinc-gaussian pulses (B₁ₚₑₐₖ=0.5 μTtₛₐₜ=0.5 s), followed by a whole-brain simultaneous multi-slice EPI readout (multi-band factor 3). A total of 27 slices with FOV of 208×208 mm² and a resolution of 2.2×2.2×4.4 mm³ were acquired using TR/TE/FA=1200 ms/17 ms/52°. 32 frequencies were acquired in 38.2 s at offsets: ±[10 (2×), 5.0, 2.5, 2.0, 1.5, 1.2, 1.0, 0.80, 0.70, 0.60, 0.50, 0.40, 0.30, 0.20, 0.10] ppm. In total 40 dynamics (Z-spectra) were acquired. Approximately five minutes into the scan (after the 8^{th} dynamic), D-Glc was administrated intravenously with a power-injector at a rate of 6.25 g/min using hospital-grade D50 glucose (D50, Hospira Inc., Lake Forest, IL; 35 g of D-Glc in 70 mL of water sterile solution prepared by the Johns Hopkins pharmacy), followed by a saline rinse. Total experiment duration was 25.5 min.

A T₁-weighted gradient-echo sequence was used for DCE MRI: TR/TE/FA=5.1 ms/2.5 ms/26°, FOV=212×212 mm², resolution=2.2×2.2×4.4 mm³. Gadoteridol (ProHance, Bracco Diagnostics, 0.1 mmol/kg) was given at a rate of 5 mL/s via a power injector followed by a saline rinse. The injection delay was 30 seconds (15 pre-contrast baseline images). Each 15-slice dynamic scan was 2.0 s and a total of 150 dynamics over 5 min was acquired. After the DCE, post-contrast MPRAGE images were obtained.

Approximately 7 min after the first GBCA injection, a second GBCA dose was injected at a 5 mL/s rate via a power injector, followed by a saline rinse. DSC was performed using single-shot EPI with TR/TE/FA=1344 ms/29 ms/90°, FOV=212×212 mm², and resolution=2.2×2.2×4.4 mm³. A 15-second injection delay (11 pre-contrast baseline images) was set. A total of 100 dynamics were acquired over 2.2 min for 25 slices.

### Post-Processing

### DS-DGE MRI

The dynamic DS spectrum signal intensities from each voxel were normalized using the average of the second of two acquisitions at ±10 ppm and then fitted to a Lorentzian line shape using the DL-based single Lorentzian fitting neural network (sLoFNet)⁴³. The linewidths, defined as FWHM in the DL fitting, were used to generate LW maps (in Hz) for each dynamic. The dynamic linewidth maps were rigid motion corrected using Elastix⁴⁸ and visually inspected for remaining motion artefacts.

A baseline was calculated by averaging the linewidth maps obtained before infusion. To remove outliers, baseline LWs greater or less than the average±2SD were discarded. LW_{base} was then calculated by averaging the remaining baseline LWs. Dynamic ΔLW images were obtained by subtracting LW_{base} from each linewidth dynamic image, LW(t), followed by normalization with LW_{base}:

$$Δ L W ( t ) ( \% ) = \frac{L W ( t ) - L {W}_{b a s e}}{L {W}_{b a s e}} \times 100 \% \qquad ( 2 )$$

Normalized area-under-the-curve (AUC) was calculated by subtracting LW_{base} from the average of dynamic LWs obtained from infusion start and through the dynamic scan (LW_{average}), followed by normalization with LW_{base}:

$$AUC ( \% ) = \frac{L {W}_{average} - L {W}_{b a s e}}{L {W}_{b a s e}} \times 100 \% \qquad ( 3 )$$

Normalized AUC over the infusion block alone was also calculated. Both fitting and calculations were performed in Python.

### DCE and DSC MRI

DCE and DSC MRI were processed using OLEA Sphere (Olea Medical Solutions, La Ciotat, France). For both sequences, motion correction was applied before the tracer kinetic modeling. The extended Toft Model was used for DCE-MRI⁴⁹ to retrieve interstitial volume (Vₑ) and the volume transfer constant (K^{trans}), a measure combining permeability and perfusion. For DSC-MRI, standard tracer kinetic modeling including leakage correction was applied to obtain leakage corrected cerebral blood volume (corr. CBV), uncorrected cerebral blood volume (uncorr. CBV) and leakage (K2)^{50,51}.

### Results

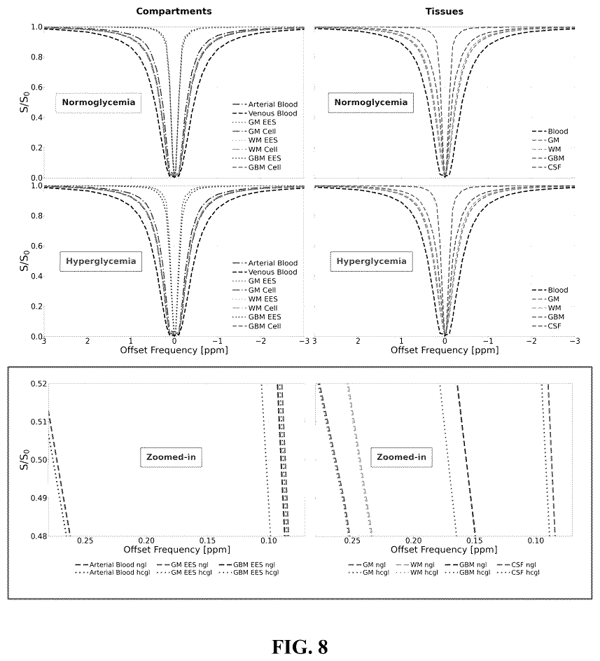

FIG. 8 shows the simulated Z-spectra for the different tissues with and without glucose infusion. Simulated baseline LWs were 87 Hz for blood, 57 Hz for arterial blood, 96 Hz for venous blood, 65 Hz for GM, 60 Hz for WM, 42 Hz for TUMOR, and 16 Hz for CSF. Simulated ΔLWs were 0.56% for blood, 1.3% for arterial blood, 0.30% for venous blood, 0.29% for GM, 0.34% for WM, 7.5% for TUMOR, and 13% for CSF.

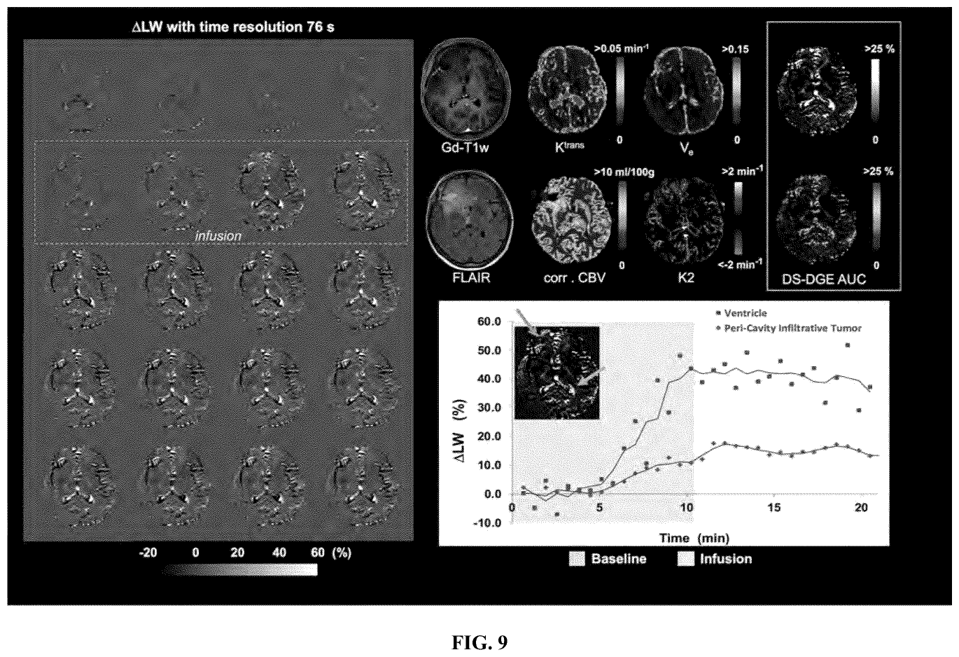

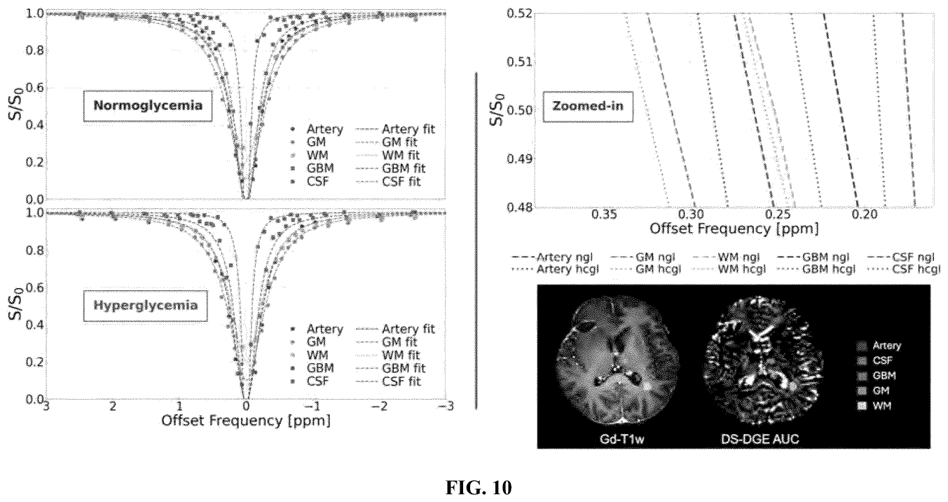

FIG. 9 shows a patient with a recurrent IDH-wildtype glioblastoma with thin peripheral contrast-enhancement (Gd-Tiw) around the resection cavity and surrounding expansile infiltrative FLAIR hyperintense tumor. Dynamic ΔLW images obtained upon infusing D-Glc are shown (averaged over two dynamics). Note the LW increases in vascular, CSF and tumor tissue. For DCE, K^{trans} shows an increase in the same regions, while Vₑ shows only a slight increase. Both K^{trans} and Vₑ are hypointense inside the cavity. The DS-DGE AUC map also displays an increase in the surrounding tumor and a hypointense core. For DSC, K2 shows enhancement comparable to DS-DGE. During the D-Glc infusion, the linewidth increased to approximately 15% for the contrast enhanced peri-cavity infiltrative tumor region and 40% for ventricular CSF. FIG. 10 shows experimental Z-spectra before and after D-Glc infusion together with DL Lorentzian fits for region-of-interests (ROIs) placed in the DS-DGE AUC, with assistance from Gd-Tiw and FLAIR images, in the anterior cerebral artery, GM, WM, tumor tissue, and CSF (ventricle). Notice that the linewidths and their broadening are of a similar order of magnitude to those simulated in FIG. 8. However, the in vivo changes in arterial blood and CSF were generally larger than those in the simulations.

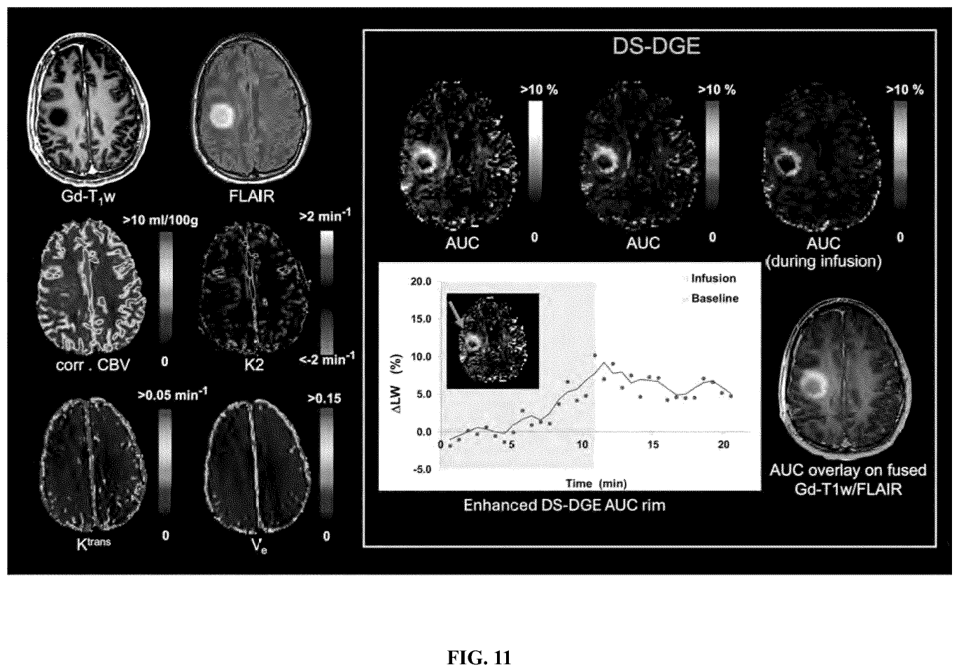

FIG. 11 shows a patient with a grade 2 IDH-mutated astrocytoma. Interestingly, the enhanced tumor rim in the DS-DGE AUC image corresponds approximately to the spatial difference between the hypointense area in Gd-Tiw and the hyperintense area in FLAIR. The enhanced areas during infusion only and over the experimental duration are of comparable size. Corrected CBV and K2 also show an increase in the corresponding area. However, K^{trans} and Vₑ appear normal.

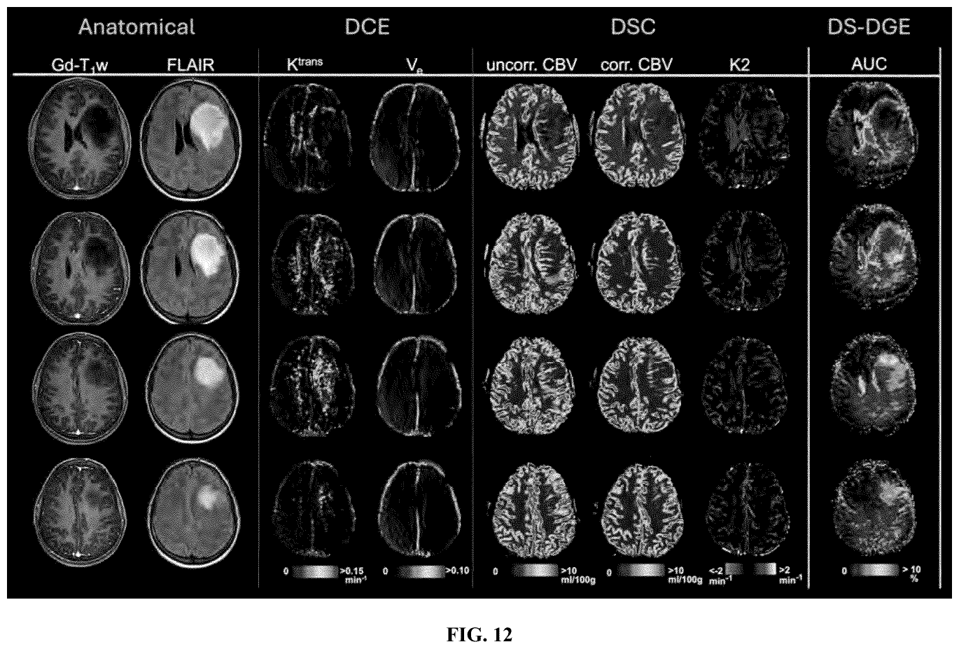

FIG. 12 shows four slices from a patient with a grade 2 IDH-mutated astrocytoma. K^{trans} shows an increase in the tumor boundary, while Vₑ shows only a slight increase. Similar to the patient in FIG. 8, the CBV lesion is strongly reduced in intensity and area after leakage correction, as reflected in the K2 enhancement. Note that DS-DGE hyperintense and hypointense tumor regions correspond approximately to the hyperintense rims and hypointense cores in the FLAIR images, respectively. The K2 images show a similar trend but over a smaller area.

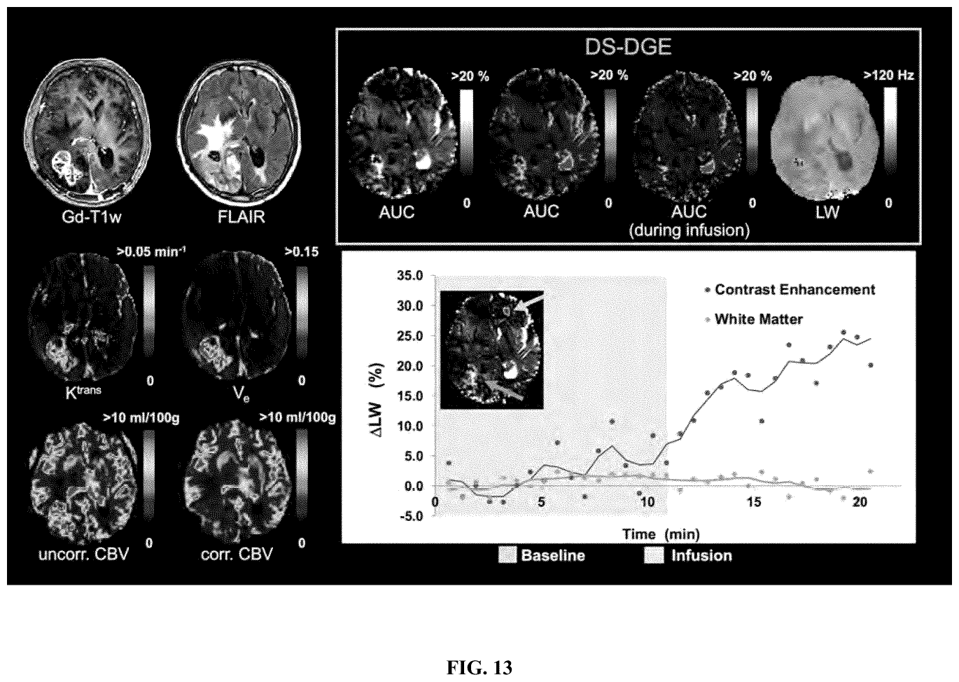

FIG. 13 shows results for a patient with ALK-mutated non-small-cell lung cancer brain metastases. K^{trans} and Vₑ are increased in the Gd-contrast-enhanced lesion area. The DS-DGE AUC map also displays an increase in part of the lesion area, whereas the white matter shows negligible LW change. Note that leakage correction strongly reduced the elevated uncorrected CBV values in the lesion area. The enhancement observed on the contralateral side in the DS-DGE map is due to ventricular CSF. The ΔLW time curve shows a continuous increase up to approximately 20% in the DS-DGE contrast-enhanced lesion, resulting in a relatively smaller lesion area enhancement in the DS-DGE AUC map calculated from the infusion block only.

### Discussion

We developed and implemented DS-DGE MRI to dynamically assess D-Glc uptake in brain tumors. This approach samples Z-spectra dynamically, which has the advantages of (i) multiple signal points per dynamic (leading to higher SNR and reduced motion sensitivity due to the Lorentzian fitting), (ii) being independent of B₀ changes in the voxel between dynamics, e.g. such as those due to motion, as the full DS spectrum is fitted and (iii) a water resonance line shape that has minimal contributions from CEST, rNOE and MTC effects and can be approximated by a Lorentzian curve^{40,42,52-54}. When applying RF saturation with low B₁, the FWHM of the DS Z-spectral line can be calculated as:

$$LW = FWHM = ( \frac{1}{π} ) \sqrt{\frac{{R}_{1} \cdot {R}_{2}^{2} + {ω}_{1}^{2} \cdot {R}_{2}}{{R}_{1}}} \approx ( \frac{1}{π} ) \sqrt{\frac{{ω}_{1}^{2} \cdot {R}_{2}}{{R}_{1}}} , \qquad ( 4 )$$

in which ω₁=γB₁ (in units of rad/s) and R_{1,2}=1/T_{1,2}. The presence of exchangeable protons at an offset from the water resonance will increase R₂^{38,39} and broaden this line shape, with relative effects expected to be largest for tissue compartments with a long water T₂. This was borne out in the simulations, where EES and CSF exhibited narrow lines and large linewidth changes when the D-Glc concentration was increased (FIG. 8). When compartments were added proportionally (Eq. 1) to generate the Z-spectra for GM, WM, and TUMOR, ΔLW was highest in TUMOR which is attributed to (i) the large concentration of D-Glc in EES after BBB breakdown, (ii) increased blood volume and EES volume, and (iii) the lower pH in tumor EES, which reduces the exchange rate and increases the exchange-based transverse relaxivity. For blood, despite having the highest D-Glc concentration, simulations showed a smaller linewidth change, which we attribute to the shorter T₂ originating from the high protein contents in both plasma (albumin) and erythrocyte (hemoglobin). Notice that an equal water-based concentration of D-Glc in plasma and erythrocytes⁵⁵⁻⁵⁷ was used, removing any effect of microvascular hematocrit on blood D-Glc concentrations. The small effects in WM and GM are attributed to the low D-Glc concentration in EES and even lower in the cells due to facilitated transport over the BBB and the cell membrane, respectively, and metabolism in the cells. The short T₂ of the cell compartment, which occupies a large volume fraction of the tissue, further reduces the DS-DGE effect size.

In patients, the effect sizes for the DS-DGE linewidth differences, ΔLW(t), were generally on the same order of magnitude as those obtained from simulations. This can be observed in FIG. 13, where the WM uptake curve shows a small increase during and after D-Glc infusion, and GM and WM intensities in the AUC maps are close to zero. The same applies to the DS-DGE AUC maps in FIGS. 9, 11, and 12. CSF had a smaller ΔLW in the simulations than sometimes observed in the experimental data (approx. 15% compared to up to 40%, FIG. 9). This difference may be due to the deviation from a Lorentzian line shape due to so-called sidebands appearing as distinct patterns when using short RF pulses at high sampling rates⁵⁸. Sidebands are prone to occur in tissues with relatively long T₂ such as CSF or other liquid environments as necrotic tumor tissue⁵⁸. For our simulations, the offset frequencies were selected to minimize sideband occurrence. In the experiments, if the same sidebands occur before and after infusion start, the linewidth change, however, may not be significantly affected. Interestingly, the maximum LW increases in blood and CSF (FIG. 9) in vivo were found to be larger than those from the simulations (FIG. 8). One potential explanation for blood could be an osmotic increase in blood water content due to the higher D-Glc concentration and a concomitant increase in T₂, previously suggested as a potential contributor to signal changes in CESL^{26,59}. Another potential explanation is that the increase in plasma blood glucose concentration, and subsequently the CSF, may be greater than what we assumed in our simulations. Previous DGE studies have reported increases up to 15 mM in plasma blood glucose^{13,30} for a maximum D-Glc dose of 25 g, while our study used 35 g. If this increase proves to be reproducible in blood, it could allow the retrieval of an arterial input function for deconvolving the tumor tissue curve, resulting in a dynamic time curve that could provide information about glucose transport and metabolism, while also removing subject variability due to insulin response. However, this is beyond the goal of this technical feasibility study.

The experimental data showed that several tumor tissues had a larger LW increase than GM and WM (FIGS. 9-13), confirming the simulations. In all four tumor cases, BBB leakage in the tumor was visible in K2 images or when comparing uncorrected CBV with corrected CBV in areas coinciding spatially with part of the DS-DGE enhancement. However, the DS-DGE enhancement area was generally larger and more pronounced in intensity, possibly reflecting the increased sensitivity to BBB disruption of the small glucose molecule and detectability at the lower pH in tumor EES. K^{trans} and Vₑ, to some degree, also showed overlap with DS-DGE enhancements, with all three parameters showing largest enhancement in the metasteses. The enhancement in tumors could differ temporally, which may also reflect different amounts of BBB disruption (e.g., FIG. 13). A rapid uptake would result in similar enhancement for both time periods (without or with post-infusion), while a slow uptake may show a difference.

These results show the feasibility for DS-DGE to outline tumor tissue with BBB disruption even before Gd-enhancement can detect it. Another important aspect is that the large effect in malignant tumor, combined with a small effect in normal tissue, constitutes an advantage over PET studies of brain malignancies, where phosphorylated ¹⁹F-deoxyglucose signal builds up in both brain tumors and healthy gray matter⁶⁰.

There are several practical considerations that need to be mentioned. First, similar to other DGE studies, our scan time was long, which increases the risk of motion, which may introduce tissue mixing, resulting in hypo- and hyperintensities in the (DS)-DGE maps^{36,37}. In addition, motion can shift the voxel to another position, resulting in erroneous ΔLW(t) curves. As motion correction can reduce these errors³⁵, we applied this to our dynamic data. However, care must be taken since interpolation errors can remove or reduce true effects. Furthermore, while motion correction was applied to the dynamic LW images, the individual frequency offset images were not corrected for motion, though these can also be affected by motion. Such a frequency-specific motion correction can be more challenging due to the intensity differences between the offsets in the dynamic Z-spectrum, especially close to the direct water saturation⁶¹. Similarly, partial volume effects due to motion can also induce intensity changes at individual frequencies. On the other hand, the Lorentzian fitting over a large number of points is performed using a pre-determined line shape, which may reduce small motion effects on the measured LW. The risk of motion can be reduced by shortening the experimental duration. However, this can introduce drawbacks such as a less robust baseline measurement and/or an incomplete glucose uptake measurement. Partial volume effects due to physiological effects, such as ventricular swelling, can also introduce signal changes³³. Assuming CSF mixes with WM at the tissue boundary, this would result in an LW reduction in WM and an LW increase in CSF. Second, this study used a D-Glc dose of 35 g (0.5 g/kg, maximum of 35 g), while previous studies have used a maximum dose of 25 g, leading to smaller effects. For example, the first DGE MRI study at 3 T by Xu et al. gave an effect size of approximately 1.5% in glioblastoma³². In a larger patient cohort, Mo et al. found effect sizes of 0.5-1.5% in low (LGG) and high grade glioma (HGG) enhancement areas using a dose of 25 g⁶². A smaller maximum dose (20 g of D-Glc in 100 mL) was used by Bender et al. in LGG and HGG patients resulting in effect sizes of maximum 0.25% in the contrast enhanced tumor area²⁹. For metastases, a recent DGE MRI study by Wu et al.³⁵ found up to a 10% increase after D-Glc infusion, while in our study, the metastases showed an increase of more than 20%. However, it is difficult to make a quantitative comparison between these studies since they differ in saturation parameters, D-Glc concentration, and contrast origin, i.e., CEST including T₂ relaxation versus linewidth change based on T₂ relaxation. In addition, these differences can be caused by individual variations of the tumor structure and properties, warranting a larger population study. Third, since we are acquiring the Z-spectra during a steady state, an additional signal decrease will occur as a function of time during the Z-spectrum acquisition. Although the frequency offsets are acquired in alternating fashion around the water frequency, this continuous signal decrease will still cause the DS to deviate from a true Lorentzian line shape. This may especially affect the narrower line shapes where fitting deviations may occur at the earlier smaller intensity drops in the Z-spectrum (FIGS. 8 and 10). Fourth, conflicting results have been reported regarding hyperglycemia's effect on perfusion, with studies showing minimal regional changes⁶³ or no effect^{64,65}. For our DCE and DSC measurements, we assumed the latter.

### CONCLUSION

We developed DS-DGE MRI to assess D-Glc uptake in brain tumors. Contrary to glucoCESL- and single-frequency glucoCEST-based DGE MRI, this approach is inherently independent of B₀ shifts occurring between the dynamics. Whole-brain dynamic Z-spectral images were acquired in less than 40 seconds, allowing imaging of D-Glc uptake curves in multiple tissues. Early patient data look highly promising, with DS-DGE highlighting lesion areas with information similar and/or complementary to Gd-based perfusion-weighted imaging. DS-DGE MRI can thus further bridge the gap between research and clinical implementation of using D-Glc as a biodegradable contrast agent.

TABLE 1
Relaxation times*, fractional volumes, D-Glc concentrations,
and water contents for tissues (b, GM, WM, TUMOR, CSF)
and their compartments (arterial, venous, EES, cell).

| | TISSUE | | | | | |
|---|---|---|---|---|---|---|
| | Arterial | Venous | | | TU- | |
| PARAMETER | Blood | Blood | GM | WM | MOR | CSF |
| T_{1, b} (s) | 1.91 | 1.73 | | | | |
| T_{1, tis}^{e} (s) | | | 3.48 | 3.48 | 3.48 | 3.48 |
| T_{1, tis}^{c} (s) | | | 1.08 | 0.65 | 1.02 | |
| T_{2, b} (s) | 0.152 | 0.052 | | | | |
| T_{2, tis}^{e} (s) | | | 2.78 | 2.78 | 2.78 | 2.78 |
| T_{2, tis}^{c} (s) | | | 0.071 | 0.055 | 0.071 | |
| f_{comp, b} (mL comp/mL | 0.30 | 0.70 | | | | |
| b) | | | | | | |
| f_{b, tis} (mL b/mL tis) | | | 0.038 | 0.018 | 0.050 | 0.00 |
| f_{e, tis} (mL EES/mL tis) | | | 0.22 | 0.22 | 0.50 | 1.00 |
| f_{c, tis} (mL cell/mL tis) | | | 0.74 | 0.76 | 0.45 | 0.00 |
| C_{b} ngl (mM) | 6.15 | 5.47 | | | | |
| C_{e, tis} ngl (mM) | | | 2.24 | 2.42 | 5.45 | 3.69 |
| C_{c, tis} ngl (mM) | | | 0.167 | 0.381 | 0.811 | |
| C_{b} hgl (mM) | 19.8 | 19.1 | | | | |
| C_{e, tis} hgl (mM) | | | 6.10 | 5.71 | 17.6 | 11.9 |
| C_{c, tis} hgl (mM) | | | 1.16 | 1.24 | 4.41 | |
| ρ_{b}^{V/V} (mL water/mL | | | 0.856 | 0.856 | 0.856 | |
| b) | | | | | | |
| ρₑ^{V/V} (mL water/mL | | | 0.938 | 0.938 | 0.938 | |
| comp) | | | | | | |
| ρ_{c}^{V/V} (mL water/mL | | | 0.809 | 0.678 | 0.674 | |
| comp) | | | | | | |
| ρₜᵢₛ^{V/V} (mL water/mL | | | 0.839 | 0.738 | 0.815 | |
| tis) | | | | | | |

*OH was set to have a T₁ of 1.20 s and T₂ of 0.100 s.
** For arterial and venous blood, a well-mixed compartment (fast exchange for water between plasma and erythrocytes) was assumed for the 500 ms saturation period. Relaxation times were calculated based on references^{66, 67}, using: https://www.kennedykrieger.org/physiologic-metabolic-anatomic-biomarkers/resources/software-and-databases/blood-t2-t1-hct-and-oxygenation-calculator and the following settings: 3 T; t_{cp} 50 ms; tₚ 50 ms; Hematocrit (Hct) 0.4; Oxygenations (Y) 0.98 and 0.60 for arterial and venous blood, respectively.
Abbreviations: f, volume fraction (fₐ + fᵥ = 1, f_{b, tis} + f_{e, tis} + f_{c, tis} = 1); C, D-Glc concentration; ρ^{V/V}, water volume density; comp, compartment; tis, tissue; a, arterial; v, venous; b, blood; e, EES; c, Cell; ngl, normoglycemia; hgl, hyperglycemia.
Values for f, C, and ρ^{V/V} are from ref. and references therein.

TABLE 2
Exclusion Criteria for the study
Exclusion Criteria

Age <18
Unwilling to participate in the study
Not capably to understand or sign inform consent
Presence of any ferromagnetic implant (cardiac pacemakers, aneurysm
clip, etc.)
Pregnancy
Claustrophobia or anxiety disorder
Diabetes mellitus (self-reported or hemoglobin A1c ≥6.5%)
eGFR documented within the last 2 weeks less than 60 mL/min/1.73 m²
Sickle cell disease
Blood iron deficiency (hemoglobin concentration <12 g/dL,
hematocrit <35%)
Multiple myeloma
Solid organ transplant
History of severe hepatic disease
Liver transplant or pending liver transplant

### REFERENCES

1. Mathur M, Jones J R, Weinreb J C. Gadolinium Deposition and Nephrogenic Systemic Fibrosis: A Radiologist's Primer. Radiographics. 2020; 40(1):153-62.
2. Woolen S A, Shankar P R, Gagnier J J, MacEachern M P, Singer L, Davenport M S. Risk of Nephrogenic Systemic Fibrosis in Patients With Stage 4 or 5 Chronic Kidney Disease Receiving a Group II Gadolinium-Based Contrast Agent: A Systematic Review and Meta-analysis. JAMA Intern Med. 2020; 180(2):223-30.
3. Gulani V, Calamante F, Shellock F G, Kanal E, Reeder S B, International Society for Magnetic Resonance in M. Gadolinium deposition in the brain: summary of evidence and recommendations. Lancet Neurol. 2017; 16(7):564-70.
4. McDonald R J, McDonald J S, Kallmes D F, Jentoft M E, Paolini M A, Murray D L, et al. Gadolinium Deposition in Human Brain Tissues after Contrast-enhanced M R Imaging in Adult Patients without Intracranial Abnormalities. Radiology. 2017; 285(2):546-54.
5. Lord M L, Chettle D R, Grafe J L, Noseworthy M D, McNeill F E. Observed Deposition of Gadolinium in Bone Using a New Noninvasive in Vivo Biomedical Device: Results of a Small Pilot Feasibility Study. Radiology. 2018; 287(1):96-103.
6. U.S. Food and Drug Administration. (2017). FDA-approved labeling for healthcare professionals: Boxed warning section. FDA document no. 105513. Retrieved from: www.fda.gov/media/105513/download. Date Accessed: Sep. 23, 2024.
7. Eichberg D G, Di L, Morell A A, Shah A H, Semonche A M, Chin C N, et al. Incidence of high grade gliomas presenting as radiographically non-enhancing lesions: experience in 111 surgically treated non-enhancing gliomas with tissue diagnosis. J Neurooncol. 2020; 147(3):671-9.
8. Marta G N, da Cunha Colombo Bonadio R R, Martins R E, Zuppani H B, de Castro G J. Cystic brain metastases in ALK-rearranged non-small cell lung cancer. Ecancermedicalscience. 2018; 12:818.
9. Falk Delgado A, Van Westen D, Nilsson M, Knutsson L, Sundgren P C, Larsson E M, et al. Diagnostic value of alternative techniques to gadolinium-based contrast agents in MR neuroimaging—a comprehensive overview. Insights Imaging. 2019; 10(1):84.
10. Ward K M, Aletras A H, Balaban R S. A new class of contrast agents for MRI based on proton chemical exchange dependent saturation transfer (CEST). J Magn Reson. 2000; 143(1):79-87.
11. Chan K W, McMahon M T, Kato Y, Liu G, Bulte J W, Bhujwalla Z M, et al. Natural D-glucose as a biodegradable MRI contrast agent for detecting cancer. Magn Reson Med. 2012; 68(6):1764-73.
12. Walker-Samuel S, Ramasawmy R, Torrealdea F, Rega M, Rajkumar V, Johnson S P, et al. In vivo imaging of glucose uptake and metabolism in tumors. Nat Med. 2013; 19(8):1067-72.
13. Xu X, Yadav N N, Knutsson L, Hua J, Kalyani R, Hall E, et al. Dynamic glucose-enhanced (DGE) MRI: translation to human scanning and first results in glioma patients. Tomography. 2015; 1(2):105-14.
14. Xu X, Chan K W, Knutsson L, Artemov D, Xu J, Liu G, et al. Dynamic glucose enhanced (DGE) MRI for combined imaging of blood-brain barrier break down and increased blood volume in brain cancer. Magn Reson Med. 2015; 74(6):1556-63.
15. Rivlin M, Navon G. Glucosamine and N-acetyl glucosamine as new CEST MRI agents for molecular imaging of tumors. Sci Rep. 2016; 6:32648.
16. Knutsson L, Xu J, Ahlgren A, van Zijl P C M. CEST, ASL, and magnetization transfer contrast: How similar pulse sequences detect different phenomena. Magn Reson Med. 2018; 80(4):1320-40.
17. Knutsson L, Xu X, van Zijl P C M, Chan K W Y. Imaging of sugar-based contrast agents using their hydroxyl proton exchange properties. NMR Biomed. 2022:e4784.
18. Jin T, Autio J, Obata T, Kim S G. Spin-locking versus chemical exchange saturation transfer MRI for investigating chemical exchange process between water and labile metabolite protons. Magn Reson Med. 2011; 65(5):1448-60.
19. Cobb J G, Xie J, Li K, Gochberg D F, Gore J C. Exchange-mediated contrast agents for spin-lock imaging. Magn Reson Med. 2012; 67(5):1427-33.
20. Yuan J, Zhou J, Ahuja A T, Wang Y X. M R chemical exchange imaging with spin-lock technique (CESL): a theoretical analysis of the Z-spectrum using a two-pool R(1rho) relaxation model beyond the fast-exchange limit. Phys Med Biol. 2012; 57(24):8185-200.
21. Zaiss M, Bachert P. Exchange-dependent relaxation in the rotating frame for slow and intermediate exchange—modeling off-resonant spin-lock and chemical exchange saturation transfer. NMR Biomed. 2013; 26(5):507-18.
22. Jin T, Mehrens H, Hendrich K S, Kim S G. Mapping brain glucose uptake with chemical exchange-sensitive spin-lock magnetic resonance imaging. J Cereb Blood Flow Metab. 2014; 34(8):1402-10.
23. Schuenke P, Paech D, Koehler C, Windschuh J, Bachert P, Ladd M E, et al. Fast and quantitative T1rho-weighted dynamic glucose enhanced MRI. Sci Rep. 2017; 7:42093.
24. Schuenke P, Koehler C, Korzowski A, Windschuh J, Bachert P, Ladd M E, et al. Adiabatically prepared spin-lock approach for T1rho-based dynamic glucose enhanced MRI at ultrahigh fields. Magn Reson Med. 2017; 78(1):215-25.
25. Paech D, Schuenke P, Koehler C, Windschuh J, Mundiyanapurath S, Bickelhaupt S, et al. T1rho-weighted dynamic glucose-enhanced MR imaging in the human brain. Radiology. 2017; 285(3):914-22.
26. Jin T, Iordanova B, Hitchens T K, Modo M, Wang P, Mehrens H, et al. Chemical exchange-sensitive spin-lock (CESL) MRI of glucose and analogs in brain tumors. Magn Reson Med. 2018; 80(2):488-95.
27. Herz K, Lindig T, Deshmane A, Schittenhelm J, Skardelly M, Bender B, et al. T1rho-based dynamic glucose-enhanced (DGErho) MRI at 3 T: method development and early clinical experience in the human brain. Magn Reson Med. 2019; 82(5):1832-47.
28. Boyd P S, Breitling J, Zimmermann F, Korzowski A, Zaiss M, Schuenke P, et al. Dynamic glucose-enhanced (DGE) MRI in the human brain at 7 T with reduced motion-induced artifacts based on quantitative R1rho mapping. Magn Reson Med. 2020; 84(1):182-91.
29. Bender B, Herz K, Deshmane A, Richter V, Tabatabai G, Schittenhelm J, et al. GLINT: GlucoCEST in neoplastic tumors at 3 T-clinical results of GlucoCEST in gliomas. MAGMA. 2022; 35(1):77-85.
30. Knutsson L, Seidemo A, Rydhog Scherman A, Markenroth Bloch K, Kalyani R R, Andersen M, et al. Arterial Input Functions and Tissue Response Curves in Dynamic Glucose-Enhanced (DGE) Imaging: Comparison Between glucoCEST and Blood Glucose Sampling in Humans. Tomography. 2018; 4(4):164-71.
31. Xu X, Xu J, Knutsson L, Liu J, Liu H, Li Y, et al. The effect of the mTOR inhibitor rapamycin on glucoCEST signal in a preclinical model of glioblastoma. Magn Reson Med. 2019; 81(6):3798-807.
32. Xu X, Sehgal A A, Yadav N N, Laterra J, Blair L, Blakeley J, et al. D-glucose weighted chemical exchange saturation transfer (glucoCEST)-based dynamic glucose enhanced (DGE) MRI at 3 T: early experience in healthy volunteers and brain tumor patients. Magn Reson Med. 2020; 84(1):247-62.
33. Seidemo A, Lehmann P M, Rydhog A, Wirestam R, Helms G, Zhang Y, et al. Towards robust glucose chemical exchange saturation transfer imaging in humans at 3 T: Arterial input function measurements and the effects of infusion time. NMR Biomed. 2022; 35(2):e4624.
34. Seidemo A, Wirestam R, Helms G, Markenroth Bloch K, Xu X, Bengzon J, et al. Tissue response curve-shape analysis of dynamic glucose-enhanced and dynamic contrast-enhanced magnetic resonance imaging in patients with brain tumor. NMR Biomed. 2023; 36(6):e4863.
35. Wu Y, Derks S, Wood T C, de Blois E, van der Veldt A A M, Smits M, et al. Improved postprocessing of dynamic glucose-enhanced CEST MRI for imaging brain metastases at 3 T. Eur Radiol Exp. 2023; 7(1):78.
36. Zaiss M, Herz K, Deshmane A, Kim M, Golay X, Lindig T, et al. Possible artifacts in dynamic CEST MRI due to motion and field alterations. J Magn Reson. 2019; 298:16-22.
37. Lehmann P M, Seidemo A, Andersen M, Xu X, Li X, Yadav N N, et al. A numerical human brain phantom for dynamic glucose-enhanced (DGE) MRI: On the influence of head motion at 3 T. Magn Reson Med. 2023; 89(5):1871-87.
38. Gore J C, Brown M S, Mizumoto C T, Armitage I M. Influence of glycogen on water proton relaxation times. Magn Reson Med. 1986; 3(3):463-6.
39. Yadav N N, Xu J, Bar-Shir A, Qin Q, Chan K W, Grgac K, et al. Natural D-glucose as a biodegradable MRI relaxation agent. Magn Reson Med. 2014; 72(3):823-8.
40. Kim M, Gillen J, Landman B A, Zhou J, van Zijl P C. Water saturation shift referencing (WASSR) for chemical exchange saturation transfer (CEST) experiments. Magn Reson Med. 2009; 61(6):1441-50.
41. Zhou Y, Bie C, van Zijl P C M, Yadav N N. The relayed nuclear Overhauser effect in magnetization transfer and chemical exchange saturation transfer MRI. NMR Biomed. 2023; 36(6):e4778.
42. Liu G, Qin Q, Chan K W, Li Y, Bulte J W, McMahon M T, et al. Non-invasive temperature mapping using temperature-responsive water saturation shift referencing (T-WASSR) MRI. NMR Biomed. 2014; 27(3):320-31.
43. Mohammed Ali S, Yadav N N, Wirestam R, Singh M, Heo H Y, van Zijl P C, et al. Deep learning-based Lorentzian fitting of water saturation shift referencing spectra in MRI. Magn Reson Med. 2023; 90(4):1610-24.
44. Herz K, Mueller S, Perlman O, Zaitsev M, Knutsson L, Sun P Z, et al. Pulseq-CEST: Towards multi-site multi-vendor compatibility and reproducibility of CEST experiments using an open-source sequence standard. Magn Reson Med. 2021; 86(4):1845-58.
45. Seidemo A, Knutsson L, Yadav N N, Wirestam R, Sundgren P C, van Zijl P C M, editors. A new kinetic model for characterization of glucose delivery, transport and metabolism for MRS and dynamic glucose enhanced (DGE) MRI of the brain. In Proceedings of the International Society of Magnetic Resonance in Medicine; 2024; Singapore.
46. Zaiss M, Anemone A, Goerke S, Longo D L, Herz K, Pohmann R, et al. Quantification of hydroxyl exchange of D-Glucose at physiological conditions for optimization of glucoCEST MRI at 3, 7 and 9.4 Tesla. NMR Biomed. 2019; 32(9):e4113.
47. Lu H, Golay X, Pekar J J, Van Zijl P C. Sustained poststimulus elevation in cerebral oxygen utilization after vascular recovery. J Cereb Blood Flow Metab. 2004; 24(7):764-70.
48. Klein S, Staring M, Murphy K, Viergever M A, Pluim J P. elastix: a toolbox for intensity-based medical image registration. IEEE Trans Med Imaging. 2010; 29(1):196-205.
49. Tofts P S. Modeling tracer kinetics in dynamic Gd-DTPA M R imaging. J Magn Reson Imaging. 1997; 7(1):91-101.
50. Knutsson L, Stahlberg F, Wirestam R. Absolute quantification of perfusion using dynamic susceptibility contrast MRI: pitfalls and possibilities. MAGMA. 2010; 23(1):1-21.
51. Knutsson L, Lindgren E, Ahlgren A, van Osch M J, Bloch K M, Surova Y, et al. Dynamic susceptibility contrast MRI with a prebolus contrast agent administration design for improved absolute quantification of perfusion. Magn Reson Med. 2014; 72(4):996-1006.
52. Mulkern R V, Williams M L. The general solution to the Bloch equation with constant rf and relaxation terms: application to saturation and slice selection. Med Phys. 1993; 20(1):5-13.
53. Smith S A, Bulte J W, van Zijl P C. Direct saturation MRI: theory and application to imaging brain iron. Magn Reson Med. 2009; 62(2):384-93.
54. Lim I A, Li X, Jones C K, Farrell J A, Vikram D S, van Zijl P C. Quantitative magnetic susceptibility mapping without phase unwrapping using WASSR. Neuroimage. 2014; 86:265-79.
55. Neely R D, Kiwanuka J B, Hadden D R. Influence of sample type on the interpretation of the oral glucose tolerance test for gestational diabetes mellitus. Diabet Med. 1991; 8(2):129-34.
56. Farrer M, Albers C J, Neil H A, Adams P C, Laker M F, Alberti K G. Assessing the impact of blood sample type on the estimated prevalence of impaired glucose tolerance and diabetes mellitus in epidemiological surveys. Diabet Med. 1995; 12(4):325-9.
57. Carstensen B, Lindstrom J, Sundvall J, Borch-Johnsen K, Tuomilehto J, Group DPSS. Measurement of blood glucose: comparison between different types of specimens. Ann Clin Biochem. 2008; 45(Pt 2):140-8.
58. Schure J R, Weinmuller S, Kamm L, Herz K, Zaiss M. Sidebands in CEST MR-How to recognize and avoid them. Magn Reson Med. 2024; 91(6):2391-402.
59. Choi W M, Chung J, Jin T, Kim S G. Effect of osmolality on dynamic glucose enhanced (DGE) MRI. 25th Annual Meeting of ISMRM; Honolulu, HI 2017. p. 194.
60. Chen W. Clinical applications of PET in brain tumors. J Nucl Med. 2007; 48(9):1468-81.
61. Simegn G L, Sun P Z, Zhou J, Kim M, Reddy R, Zu Z, et al. Motion and magnetic field inhomogeneity correction techniques for chemical exchange saturation transfer (CEST) MRI: A contemporary review. NMR Biomed. 2024:e5294.
62. Mo J, Xu X, Ma A, Lu M, Wang X, Rui Q, et al. Dynamic glucose enhanced MRI of gliomas: a preliminary clinical application. NMR Biomed. 2024; In Press.
63. Kochunov P, Wey H Y, Fox P T, Lancaster J L, Davis M D, Wang D J, et al. Changes in Cerebral Blood Flow during an Alteration in Glycemic State in a Large Non-human Primate (Papio hamadryas sp.). Front Neurosci. 2017; 11:49.
64. Xu F, Liu P, Pascual J M, Xiao G, Huang H, Lu H. Acute effect of glucose on cerebral blood flow, blood oxygenation, and oxidative metabolism. Hum Brain Mapp. 2015; 36(2):707-16.
65. Hasselbalch S G, Knudsen G M, Capaldo B, Postiglione A, Paulson O B. Blood-brain barrier transport and brain metabolism of glucose during acute hyperglycemia in humans. J Clin Endocrinol Metab. 2001; 86(5):1986-90.
66. Li W, Grgac K, Huang A, Yadav N, Qin Q, van Zijl P C. Quantitative theory for the longitudinal relaxation time of blood water. Magn Reson Med. 2016; 76(1):270-81.
67. Li W, van Zijl P C M. Quantitative theory for the transverse relaxation time of blood water. NMR Biomed. 2020; 33(5):e4207. Erratum: Quantitative theory for the transverse relaxation time of blood water. NMR Biomed. 2020; 33(8):e4329.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. For example, it is to be understood that the present disclosure contemplates that, to the extent possible, one or more features of any embodiment can be combined with one or more features of any other embodiment.

We claim:

1. A method for at least one of magnetic resonance (MR) imaging (MRI) or spectroscopy (MRS) on an MR scanner for detecting the presence of a changed amount of a substance containing exchangeable protons in one or more tissue areas in a human or non-human subject, comprising:
subjecting said subject to an MR procedure capable of generating MR signals encoding at least one tissue area in said subject in which the amount of said substance is changing;
acquiring at least one water saturation spectrum (Z-spectra) with a substantial direct water saturation (DS) component in said subject before and after a change in the amount of said substance;
detecting at least one of a tissue-based or temporal variation in a width, a shape, a frequency, or an integral of the DS component as a consequence of said change in the amount of said substance;
determining at least one tissue-related parameter from said tissue-based or temporal variation; and
ascertaining whether said at least one tissue-related parameter is abnormal.

2. The method according to claim 1, wherein said substance containing exchangeable protons is at least one of a sugar or another carbohydrate or another chemical exchange saturation transfer (CEST) agent.

3. The method according to claim 2, wherein said change in the amount of said substance containing exchangeable protons is due to one of administration of said substance to the subject or due to a physiological change in concentration of said substance induced in the subject through at least one of an intervention, a task, or a tissue-type change.

4. The method according to claim 3, wherein said at least one tissue-related parameter comprises at least one of delivery of said substance to the tissue area, uptake into that tissue area, transport of said substance into the tissue area, metabolism of said substance in the tissue area, a pass-through-speed or pass-through-amount of said substance through the tissue area, a perfusion parameter, a blood volume, a pH, or a permeability parameter.

5. The method according to claim 3, wherein said abnormality comprises at least one of a cancer, a vascular disease, an ischemia, a tissue degeneration, a tissue inflammation, or an infection.

6. The method according to claim 3, wherein said at least one tissue area comprises one of a brain, an esophagus, a breast, a pancreas, a small intestine, a colon, a lung, a rectum, a liver, a kidney, a prostate, a uterus, a testicle, a muscle, a joint, a spine, a tumor, or a bone.

7. The method according to claim 3, wherein said administration comprises one of an intravenous (i.v.) administration, an oral administration, an intraperitoneal (i.p.) administration, an intranasal administration, or an administration of a gas through breathing.

8. The method according to claim 1, wherein acquiring at least one water saturation spectrum (or Z-spectrum) with a substantial direct water saturation (DS) component includes acquiring one or more image volume elements (voxels), corresponding to a at least one of a spatial 1D, 2D or 3D map of such Z-spectra.

9. The method according to claim 8, where the Z-spectra are acquired using one or more radiofrequency field (RF) pulses with a combined total RF field strength B₁ and total RF saturation duration (tsat) that is sufficiently limited to produce a Z-spectrum dominated by the DS component, and that the DS component is sufficiently symmetric around the water frequency.

10. The method according to claim 9, in which the detecting of a temporal variation in the width, frequency, or integral of the DS spectral component as a consequence of a change in the amount of said substance is performed using at least one spectral assessment approach.

11. The method of claim 10, wherein said at least one spectral assessment approach comprises:
fitting the DS component in each Z-spectrum using methods of interpolation between the signal intensities at multiple spectral frequencies to determine width, frequency, or integral, or a combination of these;
fitting the DS component in each Z-spectrum by fitting the signal intensities at multiple spectral frequencies to a predefined shape comprising one of a Lorentzian, Gaussian, or Voight shape to determine at least one of width, frequency, or integral;
applying a Fourier transform to the Z-spectrum, baseline correcting, apodizing and zerofilling a time domain data, and fitting a resulting time-domain signal to determine at least one of signal decay rate, signal frequency, signal integral followed by determining the width from the decay rate; and
applying low-rank methods to the Z-spectrum to determine at least one of width, frequency, or integral from motion and other artifacts.

12. The method of claim 11, wherein detecting a temporal variation in the width, frequency, or integral of the DS spectral component is done by:
comparison at each time point to the normalized width, frequency or integral of the DS spectral component before said change in amount of said compound (baseline), to determine a temporal response function for said change in said compound;
determining an input function for each voxel based on the Z-spectrum before said change in amount of said compound (baseline), normalizing this input function over all data points at baseline, deconvolving the DS spectral response function, before, during and after said change in amount of said compound with this input function, to determine a temporal response function for said change in said compound;
determining an input function for each voxel based on the Z-spectrum before said change in amount of said compound (baseline), normalizing this input function over all data points at baseline, determining the center frequency of this input function, aligning the Z-spectra at all time points in terms of central frequency, subtracting this input function from the Z-spectrum at each time point to visualize the temporal change in the difference spectrum during and after said change in amount of said compound, and determining the temporal response functions for said change in said compound based on at least one of the difference signal integral or width.

13. The method of claim 12, wherein said temporal response function is used to assess tissue abnormality, using multiple approaches, comprising:
using the shape of said temporal response function in terms of at least one of rates of increase or decay, or maximum intensity,
using an area under curve (AUC) of said temporal response function, and
using individual time points of said temporal response function.

14. The method of claim 12, in which said temporal response function of the tissue is deconvolved with the temporal response function of blood water signal, a so-called arterial input function or venous input function, to derive a new temporal response function that for use to assess said tissue-related parameters and abnormalities in said tissue related parameters.

15. The method according to claim 8, wherein the Z-spectra are acquired using sufficiently high saturation field strength (B1) and length (tsat) to generate a detectable DS-component asymmetry due to a presence of fast or intermediate exchange of protons between said substance and the water.

16. The method according to claim 15, wherein the detecting of a tissue-based or temporal variation in the width, integral, or shape asymmetry of the DS spectral component as a consequence of a change in the amount of said substance is performed using at least one spectral assessment approach.

17. The method of claim 16, wherein said at least one spectral assessment approach comprises:
fitting the DS component in each Z-spectrum using existing methods of interpolation between the signal intensities at multiple spectral frequencies to determine at least one of width, integral, or asymmetry;
fitting the DS component in each Z-spectrum by fitting the signal intensities at multiple spectral frequencies to a predefined shape comprising one of a Lorentzian, Gaussian, or Voight shape to determine deviation from this shape to assess signal asymmetry;
centering the Z-spectrum, applying a Fourier transform to the Z-spectrum, baseline correcting, apodizing and zerofilling the time domain data, and fitting the resulting time-domain signal at zero frequency to determine at least one of signal decay rate, or signal integral followed by determining the width from the decay rate;
centering the Z-spectrum, comparing low and high frequency sides to assess signal asymmetry; and
applying low-rank methods to the Z-spectrum to determine at least one of width, frequency, or integral from motion and other artifacts.

18. The method of claim 17, wherein detecting a temporal variation in the width, integral, or asymmetry of the DS spectral component is done by:
comparison at each time point to the normalized width, integral, or asymmetry of the DS spectral component before said change in amount of said compound (baseline), giving a temporal response function for said change in said compound;
determining an input function for each voxel based on the Z-spectrum before said change in amount of said compound (baseline), normalizing this input function over all data points at baseline, deconvolving the DS spectral response function during and after said change in amount of said compound with this input function, to determine a temporal response function for said change in said compound;
determining an input function for each voxel based on the Z-spectrum before said change in amount of said compound (baseline), normalizing this input function over all data points at baseline, determining the center frequency of this input function, aligning the Z-spectra at all time points in terms of central frequency, subtracting this input function from the Z-spectrum at each time point to visualize the temporal change in the difference spectrum during and after said change in amount of said compound, and determining the temporal response functions for said change in said compound based on at least one of the difference signal integral, width, or the signal asymmetry relative to the central water frequency.

19. The method of claim 18, wherein said temporal response function is used to assess tissue abnormality, using multiple approaches, comprising:
using the shape of said temporal response function in terms of at least one of rates of increase or decay, or maximum intensity;
using the area under the curve (AUC) of said temporal response function; and
using individual time points of said temporal response function.

20. The method of claim 18, wherein said temporal response function of the tissue is deconvolved with at least one of a temporal response function of blood water signal, an arterial input function, or venous input function to provide a temporal response function to be used to assess said tissue-related parameters and abnormalities in said tissue related parameters.

21. The method of claim 17, wherein detecting a tissue-based variation in the width, integral, or asymmetry of the DS spectral component is done by:
comparison of the normalized width, integral, or asymmetry of the DS spectral component to that of normal tissue such as white matter or gray matter.

22. A computer-readable medium comprising non-transient computer executable code, which when executed on a computer, causes said computer to perform the method according to claim 1.

23. An MRI or MRS system comprising a processor comprising configured to perform the method of claim 1.
